(19) United States
(12) Patent Application Publication
(10) Pub. No.: US 2015/0037849 A1
(43) Pub. Date: Feb. 5, 2015

(54) MICROORGANISMS FOR THE PRODUCTION OF 5-HYDROXYTRYPTOPHAN

(71) Applicant: DANMARKS TEKNISKE UNIVERSITET, LYNGBY (DK)

(72) Inventors: Eric Michael Knight, Lyngby (DK); Jiangfeng Zhu, Kokkedal (DK); Jochen Förster, Copenhagen V (DK); Hao Luo, Vanlose (DK)

(21) Appl. No.: 14/382,152

(22) PCT Filed: Feb. 28, 2013
(86) PCT No.: PCT/EP2013/054018

§ 371 Date: Aug. 29, 2014

Related U.S. Application Data
(60) Provisional application No. 61/605,229, filed on Mar. 1, 2012.

(30) Foreign Application Priority Data
Feb. 29, 2012 (EP) 12157540.1

(51) Int. Cl. C12P 13/22; C12N 15/81; C12N 9/06; C12N 9/78; C12N 9/88; C12N 9/04; C12N 15/70; C12N 9/02

(52) U.S. Cl. CPC C12P 13/227; C12N 15/70; C12N 15/81; C12N 9/0071; C12N 9/78; C12N 9/88; C12N 9/0006; C12N 9/0028; C12Y 114/16004; C12Y 305/04016; C12Y 402/03012; C12Y 101/01153; C12Y 402/01096; C12Y 105/01034
USPC 435/108; 435/252.33; 435/254.21; 435/254.2; 435/252.3; 435/254.11; 435/257.2; 435/320.1; 435/471

(57) ABSTRACT

Recombinant microbial cells and methods for producing 5-hydroxytryptophan (5HTP) using such cells are described. More specifically, the recombinant microbial cell comprises an exogenous gene encoding an L-tryptophan hydroxylase, and means for providing tetrahydrobiopterin (THB). Related sequences and vectors for use in preparing such recombinant microbial cells are also described.

### FIELD OF THE INVENTION

The present invention relates to recombinant microorganisms and methods for producing 5-hydroxytryptophan (5HTP). More specifically, the present invention relates to a recombinant microorganism comprising a heterologous gene encoding an L-tryptophan hydroxylase, and means for providing tetrahydrobiopterin (THB), to a method of producing 5HTP comprising culturing said microorganism, to a composition comprising 5HTP obtainable by culturing said microorganism, and to uses of said composition.

### BACKGROUND OF THE INVENTION

5-hydroxy-L-tryptophan (5HTP) is a naturally occurring amino acid and chemical precursor as well as metabolic intermediate in the biosynthesis of the neurotransmitters serotonin and melatonin from tryptophan. 5HTP can be derived from the native metabolite L-tryptophan in one enzymatic step. The enzyme that catalyzes this reaction is tryptophan hydroxylase, which requires both oxygen and tetrahydropterin (THB) as cofactors. Specifically, tryptophan hydroxylase catalyzes the conversion of L-tryptophan (Schramek et al., 2001) and THB into 5-Hydroxy-L-tryptophan and 4a-hydroxytetrahydrobiopterin (HTHB). 5HTP is believed to be the transport form of 5-hydroxytryptamine (serotonin), which is produced from 5HTP by enzymatic decarboxylation. Serotonin plays a significant role as a transmitter substance in the central nervous system, and serotonin deficiency has been associated with a range of conditions, such as depression, obesity and insomnia. Dietary supplements based on 5HTP for overcoming serotonin deficiency are therefore sold in many countries. The primary source of 5HTP for such supplements is typically seeds of Griffonia simplicifolia. Extracting 5HTP from the seeds can, however, be rather costly and associated with low yields. Thus, there is a need for a simplified and more cost-effective procedure.

U.S. Pat. No. 3,830,696 describes to a process for the preparation of 5HTP by microbiologically hydroxylating L-tryptophan, D,L-tryptophan or ω-N-acyl-L-tryptophan added to the fermentation broth.

U.S. Pat. No. 3,808,101 describes a biological method of producing tryptophan and 5-substituted tryptophans, purportedly by the action of tryptophanase, by cultivation of certain microorganism strains on, e.g., indole and 5-hydroxyindole.

U.S. Pat. No. 7,807,421 B2 describes cells transformed with enzymes participating in the biosynthesis of THB and a process for the production of a biopterin compound using the same.

Winge et al. (2008), describes recombinant production of tryptophan hydroxylase (TPH2) in E. coli for subsequent purification.

### SUMMARY OF THE INVENTION

It has been found that 5-hydroxytryptophan (5HTP) can be produced in a recombinant microbial cell. Advantageously, the 5HTP can be produced from an inexpensive carbon source, providing for cost-efficient production.

The invention thus provides a recombinant microbial cell comprising an exogenous nucleic acid encoding an L-tryptophan hydroxylase, and means for providing its co-factor, THB, as well as nucleic acid vectors useful for producing such recombinant microbial cells. In some aspects, the THB is provided by one or more exogenous pathways added to the recombinant microbial cell. For example, the recombinant microbial cell may comprises an enzymatic pathway regenerating THB consumed in the L-tryptophan hydroxylase-catalyzed production of 5HTP, an enzymatic pathway producing THB from guanosin triphosphate (GTP), or both.

In other aspects, the invention provides for methods of producing 5HTP using such recombinant microbial cells, as well as for compositions comprising 5HTP produced by such recombinant microbial cells.

These and other aspects and embodiments are described in more details in the following sections.

### LEGENDS TO THE FIGURE

FIG. 1 is a schematic diagram showing exogenously added biochemical pathways for 5HTP production in a recombinant microbial cell, according to the invention. Further details are provided in Example 1.

FIG. 2 is a schematic diagram of p5HTP. Further details are provided in Example 2.

FIG. 3 shows that tryptophanase can degrade both tryptophan and 5-hydroxytryptophan in E. coli.

FIG. 4 shows HPLC chromatographs from the testing of tryptophanase activities. (a). 5-hydroxylase can be degraded in the cultures of wild type E. coli MG1655 strain to form 5-hydroxyindole. (b). E. coli MG1655 tnaA-mutant strain cannot degrade 5-hydroxytryptophan.

FIG. 5 shows a schematic diagram of pTHBDP. Further details are provided in Example 2.

FIG. 6 shows a schematic diagram of pTHB. Further details are provided in Example 2.

### DETAILED DISCLOSURE OF THE INVENTION

As described above, the present invention relates to a recombinant microbial cell capable of efficiently producing 5HTP from an exogenously added carbon source.

In a first aspect, the invention relates to a recombinant microbial cell comprising an exogenous nucleic acid sequence encoding an L-tryptophan hydroxylase (EC 1.14.16.4), and exogenous nucleic acids encoding enzymes of at least one pathway for producing THB. Such exogenous pathways include, but are not limited to, a pathway producing THB from guanosin triphosphate (GTP) and a pathway regenerating THB from 4a-hydroxytetrahydrobiopterin (HTHB). In one embodiment, the recombinant microbial cell is modified, typically mutated, to reduce tryptophan degradation, such as by reducing tryptophanase activity.

In a second aspect, the invention relates to a recombinant microbial cell of a preceding aspect or embodiment for use in a method of producing 5-hydroxytryptophan (5HTP), which method comprises culturing the microbial cell in a medium comprising a carbon source. The medium may optionally comprise THB.

In a third aspect, the invention relates to a vector comprising a nucleic acid sequence encoding an L-tryptophan hydroxylase, such as an L-tryptophane hydroxylase 1 or 2, and a nucleic acid sequence encoding one or more enzymes selected from (a) a GTP cyclohydrolase I (EC 3.5.4.16); (b) a 6-pyruvoyl-tetrahydropterin synthase (EC 4.2.3.12); (c) a sepiapterin reductase (EC 1.1.1.153); (d) a 4a-hydroxytetrahydrobiopterin dehydratase (EC 4.2.1.96); (e) a dihydropteridine reductase (EC 1.5.1.34); (f) a combination of any one or more of (a) to (e); (g) a combination of at least (b), (c) and (e), and (h) a combination of all of (a) to (e).

In a fourth aspect, the invention relates to a vector comprising nucleic acid sequences encoding an L-tryptophane hydroxylase, a 4a-hydroxytetrahydrobiopterin dehydratase; and a dihydropteridine reductase. In one embodiment, the vector further comprises nucleic acids encoding a GTP cyclohydrolase I, a 6-pyruvoyl-tetrahydropterin synthase and a sepiapterin reductase;

In a fifth aspect, the invention relates to a recombinant microbial cell transformed with a vector of the aforementioned aspects.

In a sixth aspect, the invention relates to a method of producing 5HTP, comprising culturing a recombinant microbial cell of any preceding aspect or embodiment in a medium comprising a carbon source, and, optionally, isolating 5HTP. In one embodiment, the medium does not comprise a detectable amount of exogenously added THB. In another embodiment, the medium comprises exogenously added THB.

In a seventh aspect, the invention relates to a method for preparing a composition comprising 5HTP comprising the steps of: (a) culturing a microbial cell comprising an exogenous nucleic acid encoding an L-tryptophan hydroxylase and at least one source of THB in a medium comprising a carbon source, optionally in the presence of tryptophan; (b) isolating 5-hydroxytryptophan; (c) purifying the isolated 5HTP; and (d) adding any excipients to obtain a composition comprising 5HTP. In one embodiment, the microbial cell comprises enzymes of a pathway regenerating THB from 4a-hydroxytetrahydrobiopterin. In one embodiment, the source of THB comprises exogenously added THB. In one embodiment, the source of THB comprises enzymes of a pathway producing THB from GTP.

In a eighth aspect, the invention relates to a method of producing a recombinant microbial cell, comprising transforming a microbial host cell with one or more vectors comprising nucleic acid sequences encoding (a) an L-tryptophan hydroxylase (EC 1.14.16.4); (b) a GTP cyclohydrolase I (EC 3.5.4.16); (c) a 6-pyruvoyl-tetrahydropterin synthase (EC 4.2.3.12); (d) a sepiapterin reductase (EC 1.1.1.153); (e) a 4a-hydroxytetrahydrobiopterin dehydratase (EC 4.2.1.96); and (f) a dihydropteridine reductase (EC 1.5.1.34), each one of said nucleic acid sequences being operably linked to an inducible, a regulated or a constitutive promoter, thereby obtaining the recombinant microbial cell.

In an ninth aspect, the invention relates to a composition comprising 5HTP obtainable by culturing a recombinant microbial cell comprising an exogenous nucleic acid sequence encoding an L-tryptophan hydroxylase and a source of tetrahydrobiopterin (THB) in a medium comprising a carbon source.

In a tenth aspect, the present invention relates to a use of a composition comprising 5HTP produced by a recombinant microbial cell or method described in any preceding aspect, in preparing a product such as, e.g., a dietary supplement, a pharmaceutical, a cosmceutical, a nutraceutical, a feed ingredient or a food ingredient.

### DEFINITIONS

As used herein, “exogenous” means that the referenced item, such as a molecule, activity or pathway, is added to or introduced into the host cell or microorganism. For example, an exogenous molecule can be added to or introduced into the host cell or microorganism, e.g., via adding the molecule to the media in or on which the host cell or microorganism resides. An exogenous nucleic acid sequence can, for example, be introduced either as chromosomal genetic material by integration into a host chromosome or as non-chromosomal genetic material such as a plasmid. For such an exogenous nucleic acid, the source can be, for example, a homologous or heterologous coding nucleic acid that expresses a referenced enzyme activity following introduction into the host cell or organism. Similarly, when used in reference to a metabolic activity or pathway, the term refers to a metabolic activity or pathway that is introduced into the host cell or organism, where the source of the activity or pathway (or portions thereof) can be homologous or heterologous. Typically, an exogenous pathway comprises at least one heterologous enzyme.

In the present context the term “heterologous” means that the referenced item, such as a molecule, activity or pathway, does not normally appear in the host cell or microorganism species in question.

As used herein, the terms “native” and “endogenous” means that the referenced item is normally present in or native to the host cell or microbal species in question.

As used herein, “vector” refers to any genetic element capable of serving as a vehicle of genetic transfer, expression, or replication for a exogenous nucleic acid sequence in a host cell. For example, a vector may be an artificial chromosome or a plasmid, and may be capable of stable integration into a host cell genome, or it may exist as an independent genetic element (e.g., episome, plasmid). A vector may exist as a single nucleic acid sequence or as two or more separate nucleic acid sequences. Vectors may be single copy vectors or multicopy vectors when present in a host cell. Preferred vectors for use in the present invention are expression vector molecules in which one or more functional genes can be inserted into the vector molecule, in proper orientation and proximity to expression control elements resident in the expression vector molecule so as to direct expression of one or more proteins when the vector molecule resides in an appropriate host cell.

The term “host cell” or “microbial” host cell refers to any microbial cell into which an exogenous nucleic acid sequence can be introduced and expressed, typically via an expression vector. The host cell may, for example, be a wild-type cell isolated from its natural environment, a mutant cell identified by screening, a cell of a commercially available strain, or a genetically engineered cell or mutant cell, comprising one or more other exogenous and/or heterologous nucleic acids than those of the invention.

A “recombinant cell” or “recombinant microbial cell” as used herein refers to a host cell into which one or more exogenous nucleic acid sequences of the invention have been introduced, typically via transformation of a host cell with a vector.

Unless otherwise stated, the term “sequence identity” for amino acid sequences as used herein refers to the sequence identity calculated as (n_{ref}−n_{dif})·100/n_{ref}, wherein n_{dif} is the total number of non-identical residues in the two sequences when aligned and wherein n_{ref} is the number of residues in one of the sequences. Hence, the amino acid sequence GSTDYTQNWA will have a sequence identity of 80% with the sequence GSTGYTQAWA (n_{dif}=2 and n_{ref}=10). The sequence identity can be determined by conventional methods, e.g., Smith and Waterman, (1981), Adv. Appl. Math. 2:482, by the ‘search for similarity’ method of Pearson & Lipman, (1988), Proc. Natl. Acad. Sci. USA 85:2444, using the CLUSTAL W algorithm of Thompson et al., (1994), Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., (1990), Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

Enzymes referred to herein can be classified on the basis of the handbook Enzyme Nomenclature from NC-IUBMB, 1992), see also the ENZYME site at the internet: http://www.expasy.ch/enzyme/. This is a repository of information relative to the nomenclature of enzymes, and is primarily based on the recommendations of the Nomenclature Committee of the International Union of Biochemistry and Molecular Biology (IUB-MB). It describes each type of characterized enzyme for which an EC (Enzyme Commission) number has been provided (Bairoch A. The ENZYME database, 2000, Nucleic Acids Res 28:304-305). The IUBMB Enzyme nomenclature is based on the substrate specificity and occasionally on their molecular mechanism; the classification does not in itself reflect the structural features of these enzymes.

In the present disclosure, tryptophan is of L-configuration, unless otherwise noted.

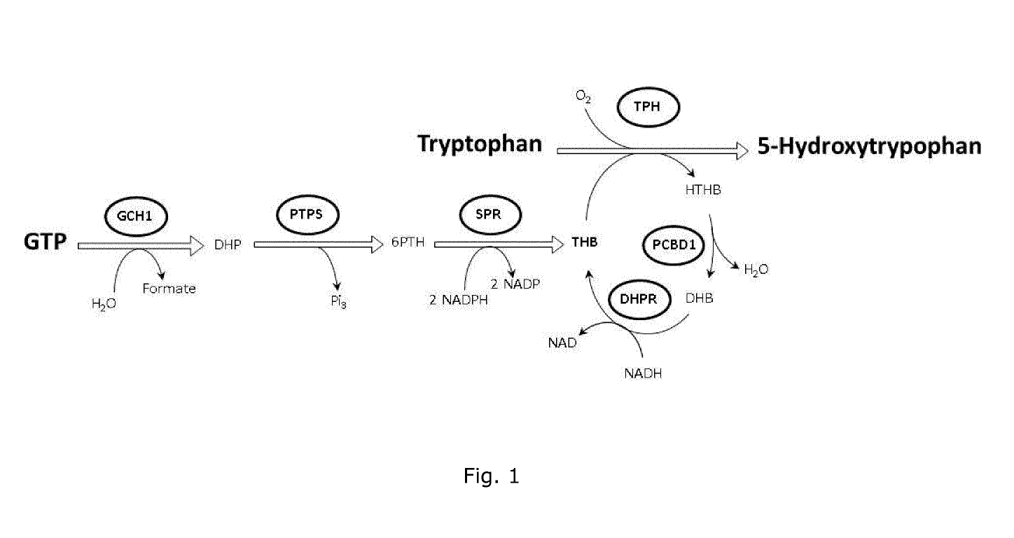

The term “substrate”, as used herein in relation to a specific enzyme, refers to a molecule upon which the enzyme acts to form a product. When used in relation to an exogenous biometabolic pathway, the term “substrate” refers to the molecule upon which the first enzyme of the referenced pathway acts, such as, e.g., GTP in the pathway shown in FIG. 1 which produces THB from GTP (see FIG. 1). When referring to an enzyme-catalyzed reaction in a microbial cell, an “endogenous” substrate or precursor is a molecule which is native to or biosynthesized by the microbial cell, whereas an “exogenous” substrate or precursor is a molecule which is added to the microbial cell, via a medium or the like.

The term “yield” as used herein means, when used regarding 5HTP production of a microbial cell, the number of moles of 5HTP per mole of the relevant carbon source in the medium, and is expressed as a percentage of the theoretical maximum possible yield.

The following are abbreviations and the corresponding EC numbers for enzymes referred to herein and in the Figures.

| Enzyme | | |
|---|---|---|
| Abbreviation | Enzyme | EC# |
| GCH1 | GTP cyclohydrolase I | EC 3.5.4.16 |
| PTPS | 6-pyruvoyl-tetrahydropterin synthase | EC 4.2.3.12 |
| SPR | sepiapterin reductase | EC 1.1.1.153 |
| DHPR | dihydropteridine reductase | EC 1.5.1.34 |
| PCBD1 | 4a-hydroxytetrahydrobiopterin dehydratase | EC 4.2.1.96 |
| TPH2 | L-tryptophan hydroxylase 2 | EC 1.14.16.4 |
| TPH1 | L-tryptophan hydroxylase 1 | EC 1.14.16.4 |

The following are abbreviations and the corresponding PubChem numbers for metabolites referred to herein and in the Figures.

| Metabolite | | |
|---|---|---|
| Abbreviation | Metabolite | PubChem# |
| GTP | guanosine triphosphate | 3346 |
| DHP | 7,8-dihydroneopterin 3′-triphosphate | 7446 |
| 6PTH | 6-pyruvoyltetrahydropterin | 6459 |
| THB | Tetrahydrobiopterin | 3570 |
| HTHB | 4a-hydroxytetrahydrobiopterin | 17396514 |
| DHB | Dihydrobiopterin | 5871 |

### SPECIFIC EMBODIMENTS OF THE INVENTION

As shown in the Examples, 5HTP can be produced in a microbial cell transformed with a tryptophane hydroxylase and exogenous pathways producing and regenerating the cofactor THB. Importantly, 5HTP production could then be achieved from a low-cost carbon source; glucose, since all required substrates for the added biosynthetic pathways were endogenously produced by the recombinant cell. Accordingly, the invention provides a recombinant microbial cell comprising an exogenous nucleic acid sequence encoding an L-tryptophan hydroxylase, and further comprises means to provide THB.

### L-Tryptophan Hydroxylase

L-tryptophan hydroxylase, also known as tryptophan 5-hydroxylase and tryptophan 5-monooxygenase, is typically classified as EC 1.14.16.4, and converts the substrate L-tryptophan to 5HTP in the presence of its cofactors THB and oxygen, as shown in FIG. 1.

Sources of nucleic acid sequences encoding an L-tryptophan hydroxylase include any species where the encoded gene product is capable of catalyzing the referenced reaction, including humans, mammals such as, e.g., mouse, cow, horse, chicken and pig, as well as other animals. In humans and, it is believed, in other mammals, there are two distinct TPH alleles, referred to herein as TPH1 and TPH2, respectively. Exemplary nucleic acids encoding L-tryptophan hydroxylase for use in aspects and embodiments of the present invention include, but are not limited to, those encoding Oryctolagus cuniculus (rabbit) TPH1 (SEQ ID NO:1); human TPH1 (SEQ ID NO:2; UniProt P17752-2), human TPH2 (SEQ ID NO:3; UniProt P17752-1) as well as those encoding L-tryptophan hydroxylase from Bos taurus (cow, SEQ ID NO:4), Sus scrofa (pig, SEQ ID NO:5), Gallus gallus (SEQ ID NO:6), Mus musculus (mouse, SEQ ID NO:7) and Equus caballus (horse, SEQ ID NO:8), as well as variants, homologs or active fragments thereof. In one embodiment, the nucleic acid encodes SEQ ID NO:1, or a variant, homolog or catalytically active fragment thereof.

In one embodiment, the nucleic acid sequence encodes an L-tryptophane hydroxylase which is a variant or homolog of any one or more of the aforementioned L-tryptophane hydroxylases, having L-tryptophan hydroxylase activity and a sequence identity of at least 30%, such as at least 50%, such as at least 60%, such as at least 70%, such as at least 80%, such as at least 90%, such as at least 95%, such as at least 99%, over at least the catalytically active portion, optionally the full-length, of a reference amino acid sequence selected from any one or more of SEQ ID NOS:1 to 9. For example, the sequence identify between the human TPH1 and TPH2 enzymes is about 65%. The variant or homolog may comprise, for example, 2, 3, 4, 5 or more, such as 10 or more, amino acid substitutions, insertions or deletions as compared to the reference amino acid sequence. In particular conservative substitutions are considered. These are typically within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In: The Proteins, Academic Press, New York. The most commonly occurring exchanges are Ala to Ser, Val to Ile, Asp to Glu, Thr to Ser, Ala to Gly, Ala to Thr, Ser to Asn, Ala to Val, Ser to Gly, Tyr to Phe, Ala to Pro, Lys to Arg, Asp to Asn, Leu to Ile, Leu to Val, Ala to Glu, and Asp to Gly. For example, homologs, such as orthologs or paralogs, to TPH1 or TPH2 having L-tryptophan hydroxylase activity can be identified in the same or a related mammalian or other animal species using the reference sequences provided and appropriate activity tests. Assays for measuring L-tryptophan hydroxylase activity in vitro are well-known in the art (see, e.g., Winge et al. (2008), Biochem. J., 410, 195-204 and Moran, Daubner, & Fitzpatrick, 1998). With the complete genome sequences now available for hundreds of species, most of which available via public databases such as NCBI, the identification of homologous genes encoding the requisite biosynthetic activity in related or distant species, the interchange of genes between organisms is routine and well known in the art.

In one embodiment, the nucleic acid sequence encoding an L-tryptophan hydroxylase encodes a fragment of one of the full-length L-tryptophan hydroxylases, variants or homologs described herein, which fragment has L-tryptophan hydroxylase activity. Notably, the TPH1 used in Examples 2-4 was a double truncated TPH1 where both the regulatory and interface domains of the full-length enzyme (SEQ ID NO:1) had been removed so that only the catalytic core of the enzyme remained, to increase heterologous expression in E. coli and the stability of the enzyme (Moran, Daubner, & Fitzpatrick, 1998). Specifically, the truncation resulted in a fragment corresponding to amino acids Met102 to Ser416 of the full-length enzyme. Accordingly, in one embodiment, the nucleic acid sequence encoding the L-tryptophan hydroxylase encodes the catalytic core of a naturally occurring L-tryptophan hydroxylase or a variant thereof. The fragment may, for example, correspond to Met102 to Ser416 of any one of SEQ ID NOS:2 to 8 or a variant or homolog thereof, when aligned with SEQ ID NO:1. In a particular embodiment, the nucleic acid sequence encodes the sequence of the catalytical core of Oryctolagus cuniculus TPH1, SEQ ID NO:9, or a variant thereof. In another particular embodiment, the nucleic acid sequence comprises the sequence of SEQ ID NO:40.

In the recombinant host cell, the L-tryptophan hydroxylase is typically sufficiently expressed so that an increased level of 5HTP production from L-tryptophan can be detected as compared to the microbial host cell prior to transformation with the L-tryptophan hydroxylase, or to another suitable control. Exemplary assays for measuring the level of 5HTP production from L-tryptophan is provided in Examples 4 and 5. In these Examples, the recombinant strain tested also comprised exogenous pathways for producing and regenerating the co-factor, THB. However, for testing L-tryptophan hydroxylase activity or for actual production of 5HTP, the THB can additionally or alternatively be added to the culture medium at a suitable concentration, for example at a concentration of about 0.1 μM or higher, such as from about 0.01, 0.02, 0.05, or 0.1 mM to about 0.1, 0.25, 1, or 10 mM, such as, e.g., from about 0.02 to about 2 mM, such as from about 0.05 to about 0.25 mM. In one exemplary embodiment, a recombinant microbial cell comprising a tryptophane hydroxylase produces at least 5%, such as at least 10%, such as at least 20%, such as at least 50%, such as at least 100% or more 5HTP than the corresponding host cell from L-tryptophan which is added to the culture medium at a suitable concentration, e.g., in the range 0.1 to 50 g/L, such as in the range of 0.2 to 10 g/L, or which is endogenously produced from a carbon source. Optionally, the host cell may be one that already has an endogenous capability for producing 5HTP, see, e.g., U.S. Pat. No. 3,808,101, U.S. Pat. No. 3,830,696 and references cited therein, reporting that some microbial strains (e.g., Proteus mirabilis (ATCC 15290) and Bacillus subtilis (ATCC 21733)) were capable of producing 5HTP from fermentation of a substrate such as 5-hydroxyindole or L-tryptophan.

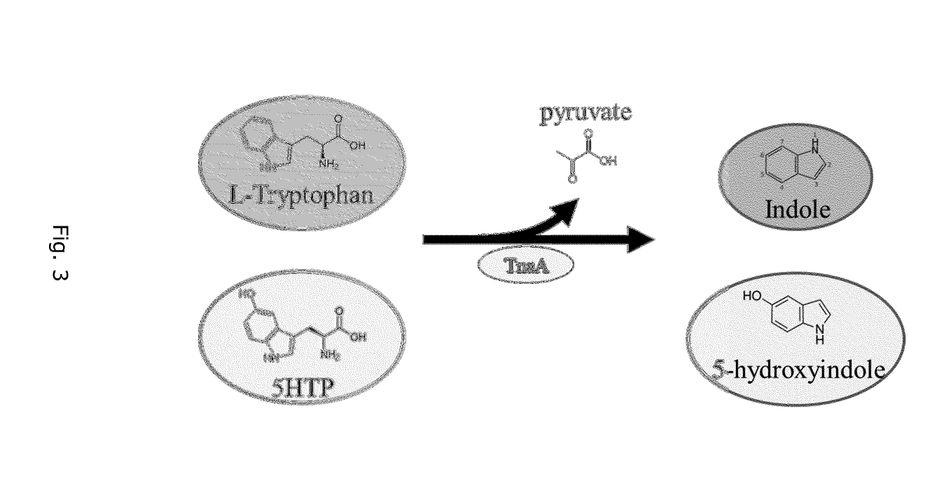

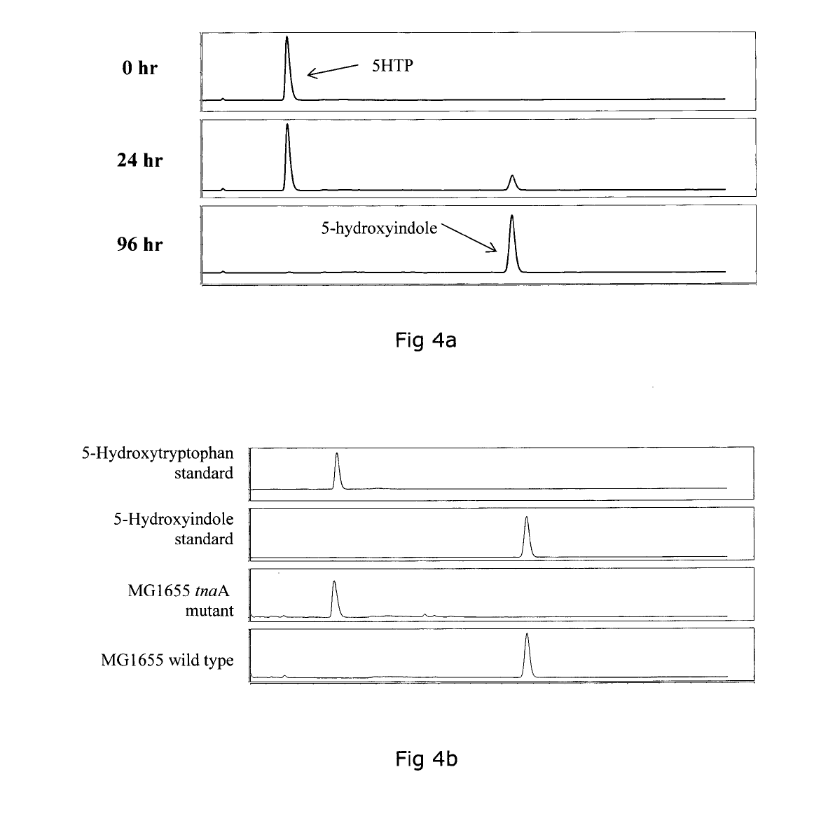

In one embodiment, the microbial cell is modified, typically mutated, to reduce tryptophanase activity. Tryptophanase or tryptophan indole-lyase (EC 4.1.99.1), encoded by the tnaA gene in E. coli, catalyzes the hydrolytic cleavage of L-tryptophan to indole, pyruvate and NH₄⁺. Active tryptophanase consists of four identical subunits, and enables utilization of L-tryptophan as sole source of nitrogen or carbon for growth together with a tryptophan transporter encoded by tnaC gene. Tryptophanase is a major contributor towards the cellular L-cysteine desulfhydrase (CD) activity. In vitro, tryptophanase also catalyzes α, β elimination, β replacement, and α hydrogen exchange reactions with a variety of L-amino acids (Watanabe, 1977). As shown in Example 5, E. coli tryptophanase can degrade also 5HTP, thus reducing the yield of 5HTP (FIGS. 3 and 4). Tryptophan degradation mechanisms are known to also exist in other microorganisms. For instance, in S. cerevisiae, there are two different pathways for the degradation of tryptophan (The Erlich pathway and the kynurenine pathway, respectively), involving in their first step the ARO8, ARO9, ARO10, and/or BNA2 genes. Reducing tryptophan degradation, such as by reducing tryptophanase activity, can be achieved by, e.g., a site-directed mutation in or deletion of a gene encoding a tryptophanase, such as the tnaA gene (in E. coli or other organisms such as Enterobacter aerogenes), or kynA gene (in Bacillus species), or one or more of the ARO8, ARO9, ARO10 and BNA2 genes (in S. cerevisiae). Alternatively, tryptophanase activity can be reduced reducing the expression of the gene by introducing a mutation in, e.g., a native promoter element, or by adding an inhibitor of the tryptophanase.

### Tetrahydrobiopterin

The recombinant microbial cell of the invention further comprises means to provide or produce THB, such as exogenous nucleic acids encoding at least one pathway for producing THB. THB is native to most animals, where it is biosynthesized from GTP. However, while THB has been found in some lower eukaryotes such as fungi and in particular groups of bacteria such as, e.g., cyanobacteria and anaerobic photosynthetic bacteria of Chlorobium species, its presence in microbes is believed to be rare. For example, THB is not native to E. coli or S. cerevisiae. Accordingly, for aspects and embodiments of the invention where THB is not added to the recombinant cells or not efficiently produced by the microbial host cell itself, THB production capability must be added. For example, the recombinant microbial cell can comprise exogenous nucleic acids encoding enzymes of a pathway producing THB from GTP and/or a pathway regenerating THB from HTHB.

First THB Pathway—THB Production from GTP

In one embodiment, the recombinant cell comprises a pathway producing THB from GTP and herein referred to as “first THB pathway”, comprising a GTP cyclohydrolase I (GCH1), a 6-pyruvoyl-tetrahydropterin synthase (PTPS), and a sepiapterin reductase (SPR) (see FIG. 1). The addition of such a pathway to microbial cells such as E. coli (3M101 strain), S. cerevisiae (KA31 strain) and Bacillus subtilis (1A1 strain (TrpC2)) has been described, see, e.g., Yamamoto (2003) and U.S. Pat. No. 7,807,421, which are hereby incorporated by reference in their entireties.

The GCH1 is typically classified as EC 3.5.4.16, and converts GTP to DHP in the presence of its cofactor, water, as shown in FIG. 1. Sources of nucleic acid sequences encoding a GCH1 include any species where the encoded gene product is capable of catalyzing the referenced reaction, including humans, mammals such as, e.g., mouse, as well as microbial GCH1 enzymes. Exemplary nucleic acids encoding GCH1 enzymes for use in aspects and embodiments of the present invention include, but are not limited to, those encoding human GCH1 (SEQ ID NO:10), GCH1 from Mus musculus (SEQ ID NO:11), E. coli (SEQ ID NO:12), S. cerevisiae (SEQ ID NO:13), Bacillus subtilis (SEQ ID NO:14), Streptomyces avermitilis (SEQ ID NO:15), and Salmonella typhi (SEQ ID NO:16), as well as variants, homologs and catalytically active fragments thereof. In some embodiments, the microbial host cell endogenously comprises sufficient amounts of a native GCH1. In these cases transformation of the host cell with an exogenous nucleic acid encoding a GCH1 is optional. In other embodiments, the exogenous nucleic acid encoding a GCH1 can encode a GCH1 which is endogenous to the microbial host cell, e.g., in the case of host cells such as E. coli, S. cerevisiae, Bacillus subtilis and Streptomyces avermitilis. In E. coli, for example, the expression of the GCH1 gene is regulated by the SoxS system. Should higher levels of GCH1 be needed, GCH1 from E. coli or another suitable source can be provided exogenously. In a particular embodiment, the exogenous nucleic acid sequence encodes E. coli GCH1, SEQ ID NO:12. In another particular embodiment, the nucleic acid sequence comprises the sequence of SEQ ID NO:41.

The PTPS is typically classified as EC 4.2.3.12, and converts DHP to 6PTH, as shown in FIG. 1. Sources of nucleic acid sequences encoding a PTPS include any species where the encoded gene product is capable of catalyzing the referenced reaction, including human, mammalian and microbial species. Exemplary nucleic acids encoding PTPS enzymes for use in aspects and embodiments of the present invention include, but are not limited to, those encoding human PTPS (SEQ ID NO:17), rat PTPS (SEQ ID NO:18), and PTPS from Bacteroides thetaiotaomicron (SEQ ID NO:19), Thermosynechococcus elongates (SEQ ID NO:20), Streptococcus thermophilus (SEQ ID NO:21), and Acaryochloris marina (SEQ ID NO:22), as well as variants, homologs and catalytically active fragments thereof. In some embodiments, the microbial host cell endogenously comprises a sufficient amount of a native PTPS. In these cases transformation of the host cell with an exogenous nucleic acid encoding a PTPS is optional. In other embodiments, the exogenous nucleic acid encoding a PTPS can encode a PTPS which is endogenous to the microbial host cell, e.g., in the case of host cells such as Streptococcus thermophilus. In a particular embodiment, the exogenous nucleic acid sequence encodes rat PTPS, SEQ ID NO:18. In another particular embodiment, the nucleic acid sequence comprises the sequence of rat PTPS, SEQ ID NO:42.

The SPR is typically classified as EC 1.1.1.153, and converts 6PTH to THB in the presence of its cofactor NADPH, as shown in FIG. 1. Sources of nucleic acid sequences encoding an SPR include any species where the encoded gene product is capable of catalyzing the referenced reaction, including humans, mammalian species such as cow, rat and mouse, and other animals. Exemplary nucleic acids encoding SPR enzymes for use in aspects and embodiments of the present invention include, but are not limited to, those encoding human SPR (SEQ ID NO:23), and SPR from rat (SEQ ID NO:24), mouse (SEQ ID NO:25), cow (SEQ ID NO:26), Danio rerio (Zebrafish, SEQ ID NO:27) and Xenopus laevis (African clawed frog, SEQ ID NO:28), as well as variants, homologs and catalytically active fragments thereof. Typically, the exogenous nucleic acid encoding an SPR is heterologous to the host cell. In a particular embodiment, the exogenous nucleic acid encodes rat SPR, SEQ ID NO:24. In another particular embodiment, the nucleic acid sequence comprises the sequence of SEQ ID NO:43.

In specific embodiments, one or more of the exogenous nucleic acids encoding GCH1, PTPS and SPR enzymes encodes a variant or homolog of any one or more of the aforementioned GCH1, PTPS and SPR enzymes, having the referenced activity and a sequence identity of at least 30%, such as at least 50%, such as at least 60%, such as at least 70%, such as at least 80%, such as at least 90%, such as at least 95%, such as at least 99%, over at least the catalytically active portion, optionally the full length, of the reference amino acid sequence. The variant or homolog may comprise, for example, 2, 3, 4, 5 or more, such as 10 or more, amino acid substitutions, insertions or deletions as compared to the reference amino acid sequence. In particular conservative substitutions and/or amino acid substitutions which do not alter specific activity are considered. Homologs, such as orthologs or paralogs, to GCH1, PTPS or SPR and having the desired activity can be identified in the same or a related animal or microbial species using the reference sequences provided and appropriate activity testing.

In the recombinant host cell, the enzymes of the first THB pathway are typically sufficiently expressed in sufficient amounts to detect an increased level of 5HTP production from L-tryptophan as compared to the recombinant microbial cell without transformation with these enzymes (i.e., the recombinant cell comprising only L-tryptophan hydroxylase), or to another suitable control. Exemplary assays for measuring the level of 5HTP production from L-tryptophan is provided in Examples 4 and 5. In one exemplary embodiment, the recombinant microbial cell produces at least 5%, such as at least 10%, such as at least 20%, such as at least 50%, such as at least 100% or more 5HTP than the recombinant cell without transformation with GCH1, PTPS and/or SPR enzymes. Alternatively, the expression and activity of the enzymes of the first THB pathway, i.e., production of THB or related products, can be tested according to methods described in Yamamoto (2003), U.S. Pat. No. 7,807,421, or Woo et al. (2002), Appl. Environ. Microbiol. 68, 3138, or other methods known in the art.

### Second THB Pathway—THB Regeneration

In one embodiment, the recombinant cell comprises a pathway producing THB by regenerating THB from HTHB, herein referred to as “second THB pathway”, comprising a 4a-hydroxytetrahydrobiopterin dehydratase (PCBD1) and a 6-pyruvoyl-tetrahydropterin synthase (DHPR). As shown in FIG. 1, the second THB pathway converts the HTHB formed by the L-tryptophan hydroxylase-catalyzed hydroxylation of L-tryptophan back to THB, thus allowing for a more cost-efficient 5HTP production.

The PCBD1 is typically classified as EC 4.2.1.96, and converts HTHB to DHB in the presence of water, as shown in FIG. 1. Sources of nucleic acid sequences encoding a PCBD1 include any species where the encoded gene product is capable of catalyzing the referenced reaction, including microbial species. Exemplary nucleic acids encoding GCH1 enzymes for use in aspects and embodiments of the present invention include, but are not limited to, those encoding PCBD1 from Pseudomonas aeruginosa (SEQ ID NO:29), Bacillus cereus var. anthracis (SEQ ID NO:30), Corynebacterium genitalium (ATCC 33030) (SEQ ID NO:31), Lactobacillus ruminis ATCC 25644 (SEQ ID NO:32), and Rhodobacteraceae bacterium HTCC2083 (SEQ ID NO:33), as well as variants, homologs and catalytically active fragments thereof. In some embodiments, the microbial host cell endogenously comprises a sufficient amount of a native PCBD1. In these cases, transformation of the host cell with an exogenous nucleic acid encoding a PCBD1 is optional. In other embodiments, the exogenous nucleic acid encoding a PCBD1 can encode a PCBD1 which is endogenous to the microbial host cell, e.g., in the case of host cells from Bacillus cereus, Corynebacterium genitalium, Lactobacillus ruminis or Rhodobacteraceae bacterium. In a particular embodiment, the exogenous nucleic acid sequence encodes Pseudomonas aeruginosa PCBD1, SEQ ID NO:29. In another particular embodiment, the nucleic acid sequence comprises the sequence of SEQ ID NO:44.

The DHPR is typically classified as EC 1.5.1.34, and converts DHB to THB in the presence of cofactor NADH, as shown in FIG. 1. Sources of nucleic acid sequences encoding a DHPR include any species where the encoded gene product is capable of catalyzing the referenced reaction, including humans and other mammalian species such as rat, pig, and microbial species. Exemplary nucleic acids encoding DHPR enzymes for use in aspects and embodiments of the present invention include, but are not limited to, those encoding DHPR from human (SEQ ID NO:34), rat (SEQ ID NO:35), pig (SEQ ID NO:36) cow (SEQ ID NO:37), E. coli (SEQ ID NO:38), Dictyostelium discoideum (SEQ ID NO:39), as well as variants, homologs or catalytically active fragments thereof. In a particular embodiment, the exogenous nucleic acid encodes E. coli DHPR, SEQ ID NO:38. In another particular embodiment, the nucleic acid sequence comprises the sequence of SEQ ID NO:45.

In specific embodiments, one or more of the exogenous nucleic acids encoding PCBD1 and DHPR enzymes encodes a variant or homolog of any one or more of the aforementioned PCBD1 and DHPR enzymes, having the referenced activity and a sequence identity of at least 30%, such as at least 50%, such as at least 60%, such as at least 70%, such as at least 80%, such as at least 90%, such as at least 95%, such as at least 99%, over at least the catalytically active portion, optionally the full length of the reference amino acid sequence.

The variant or homolog may comprise, for example, 2, 3, 4, 5 or more, such as 10 or more, amino acid substitutions, insertions or deletions as compared to the reference amino acid sequence. In particular conservative substitutions and/or amino acid substitutions which do not alter specific activity are considered. Homologs, such as orthologs or para logs, to PCBD1 or DHPR and having the desired activity can be identified in the same or a related animal or microbial species using the reference sequences provided and appropriate activity testing.

In the recombinant host cell, the enzymes of the second THB pathway are typically sufficiently expressed so that an increased level of 5HTP production from L-tryptophan can be detected as compared to the recombinant microbial cell without transformation with these enzymes (i.e., the recombinant cell comprising only L-tryptophan hydroxylase) in the presence of a THB source, or to another suitable control. Exemplary assays for measuring the level of 5HTP production from L-tryptophan is provided in Examples 4 and 5. In one exemplary embodiment, the recombinant microbial cell produces at least 5%, such as at least 10%, such as at least 20%, such as at least 50%, such as at least 100% or more 5HTP than the recombinant cell without transformation with PCBD1 and DHPR enzymes.

### Combination of First and Second Pathway

As shown in FIG. 1, a successful combination of both the first and second THB pathways in the recombinant cell, introducing pathways for producing THB from GTP and for regenerating THB consumed by L-tryptophan hydroxylase, is especially advantageous. Thereby, the addition of THB, as well as the addition of L-tryptophan, can be avoided, allowing for 5HTP production from an inexpensive carbon source. As shown in Example 5, 5HTP production was obtained in a recombinant E. coli strain (comprising both the first and second THB pathways) in LB medium supplemented with glucose and/or L-tryptophan. In M9 medium, supplementation with tryptophan produced the highest 5HTP measurements. Accordingly, in one embodiment, the invention provides for recombinant microbial cells, processes and methods where the recombinant host cell comprises both the first and second pathways of any preceding aspect or embodiment.

### Vectors

The invention also provides a vector comprising a nucleic acid sequence encoding an L-tryptophan hydroxylase as described in any preceding embodiment, and a nucleic acid sequence encoding one or more enzymes of the first and/or second THB pathways, as described in any preceding embodiment and as shown in FIG. 1. The specific design of the vector depends on whether the intended microbial host cell is to be provided with one or both THB pathways, as well as on whether host cell endogenously produces sufficient amounts of one or more of the enzymes of the THB pathways. For example, for an E. coli host cell, it may not be necessary to include a nucleic acid sequence encoding a GCH1, since the enzyme is native to E. coli. Additionally, for transformation of a particular host cell, two or more vectors with different combinations of the enzymes used in the present invention can be applied.

The vector may, for example, comprise a nucleic acid sequence encoding an L-tryptophan hydroxylase and one or more enzymes of the first THB pathway. In one embodiment, the nucleic acid encodes an SPR, and optionally one or both of a GCH1 and a PTPS. In one embodiment, the vector comprises a nucleic acid sequence encoding an SPR and a PTPS, and optionally a GCH1. In one embodiment, the nucleic acid encodes an SPR, a PTPS and a GCH1. Examples of nucleic acids encoding each of these enzymes are provided herein, and specifically include variants, homologues and catalytically active fragments thereof.

Also or alternatively, the vector may, for example, comprise a nucleic acid sequence encoding an L-tryptophan hydroxylase and one or both enzymes of the second THB pathway. In one embodiment, the nucleic acid encodes a DHPR, and optionally a PCBD1. In one embodiment, the vector comprises a nucleic acid sequence encoding a DHPR and a PCBD1. Examples of nucleic acids encoding each of these enzymes are provided herein, and specifically include variants, homologues and catalytically active fragments thereof.

In one embodiment, the vector comprises a nucleic acid sequence encoding an L-tryptophan hydroxylase, an SPR and a DHPR, and optionally a GCH1, a PTPS, a PCBD1 or a combination of any thereof. In one embodiment, the vector comprises a nucleic acid sequence encoding an L-tryptophan hydroxylase, an SPR and a DHPR, and a combination of at least two of a GCH1, a PTPS, and a PCBD1.

The vector can be a plasmid, phage vector, viral vector, episome, an artificial chromosome or other polynucleotide construct, and may, for example, include one or more selectable marker genes and appropriate regulatory control sequences.

Regulatory control sequences are operably linked to the encoding nucleic acid sequences, and include constitutive, regulatory and inducible promoters, transcription enhancers, transcription terminators, and the like which are well known in the art. The encoding nucleic acid sequences can be operationally linked to one common expression control sequence or linked to different expression control sequences, such as one inducible promoter and one constitutive promoter.

The procedures used to ligate the various regulatory control and marker elements with the encoding nucleic acid sequences to construct the vectors of the present invention are well known to one skilled in the art (see, e.g., Sambrook et al., 2001, supra). In addition, methods have recently been developed for assembling of multiple overlapping DNA molecules (Gibson et al., 2008) (Gibson et al., 2009) (Li & Elledge, 2007), allowing, e.g., for the assembly multiple overlapping DNA fragments by the concerted action of an exonuclease, a DNA polymerase and a DNA ligase.

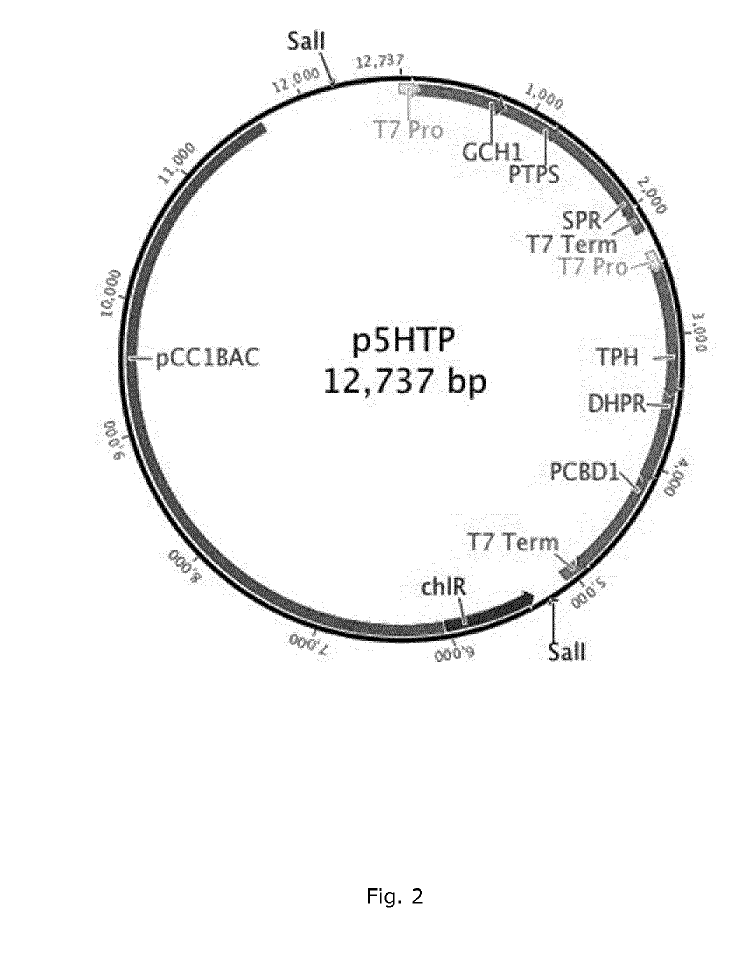

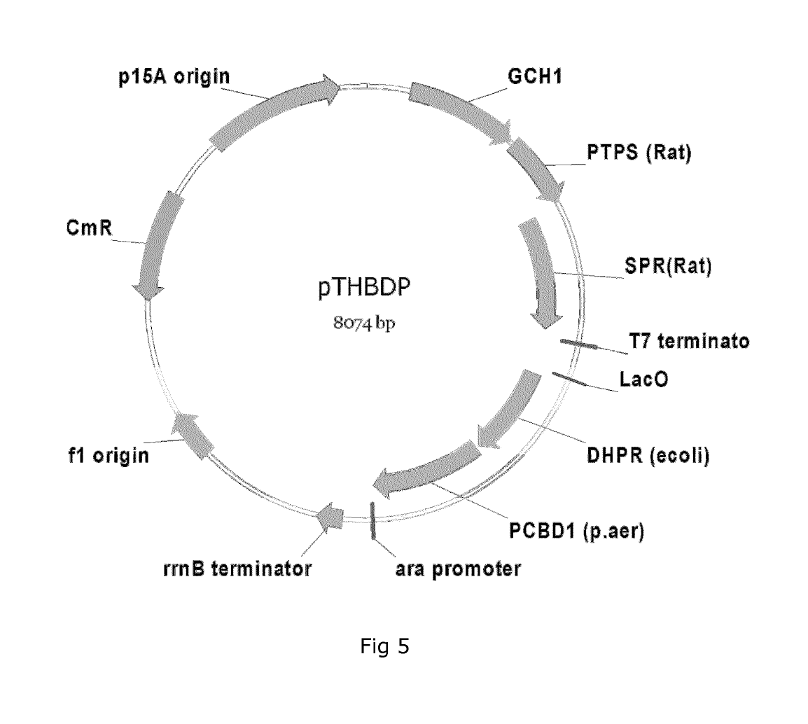

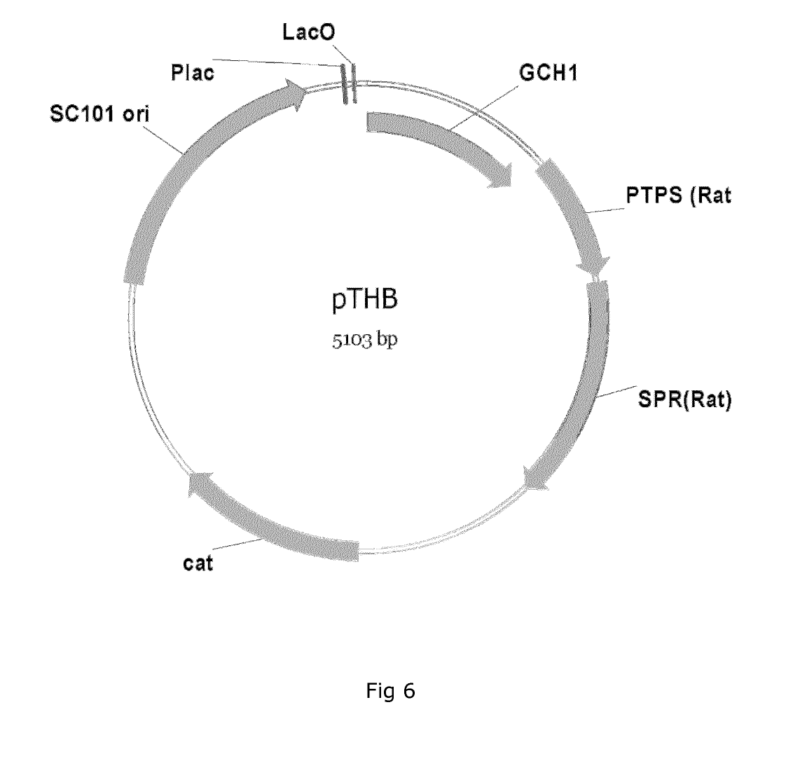

Example 2 describes the construction of a 12,737 bp BAC comprising nucleic acid sequences encoding a GCH1, a PTPS, an SPR, a TPH1, a DHPR, and a PCBD1, all under the control of a single promoter (T7 RNA polymerase). Example 2 also describes the construction of pTHB and pTHBDP vectors comprising some of these components but under the control of lac promoter. These are schematically depicted in FIGS. 6 and 5, respectively. Accordingly, in one embodiment, the vector of the invention may comprise (a) a nucleic acid sequence encoding an L-tryptophan hydroxylase, (b) nucleic acid sequences encoding one or more enzymes of the first and/or second THB pathways, as described in any preceding embodiment, (c) regulatory control sequences such as, e.g., promoter and termination sequences, and (d) one or more marker genes. In one embodiment, these elements are arranged in the order shown in FIG. 2, which is a schematic description of plasmid p5HTP. In one embodiment, the vector comprises the components of any one of pTHB, pTHBDP or pTRP, as described in Example 2, optionally in the same order as in pTHB, pTHBDP or pTRP, respectively. For example, the vector may comprise nucleic acid sequences corresponding to (a) an L-tryptophan hydroxylase and GCH1, PTPS, and SPR enzymes, one or more ribosomal binding sites, and T7 or lac promoter and T7-terminator, or (b) an L-tryptophan hydroxylase, PCBD1 and DHPR enzymes, one or more ribosomal binding sites, and T7 or lac promoter and T7-terminator. In one embodiment, the vector comprises the nucleic acid sequence of any one of pTHB (SEQ ID NO:51 or 93), pTHBDP (SEQ ID NO:92), pTRP (SEQ ID NO:52) or p5HTP (SEQ ID NO:61).

The promoter sequence is typically one that is recognized by the intended host cell. For an E. coli host cell, suitable promoters include, but are not limited to, the lac promoter, the T7 promoter, pBAD, the tet promoter, the Lac promoter, the Trc promoter, the Trp promoter, the recA promoter, the λ (lamda) promoter, and the PL promoter. For Streptomyces host cells, suitable promoters include that of Streptomyces coelicolor agarase (dagA). For a Bacillus host cell, suitable promoters include the sacB, amyL, amyM, amyQ, penP, xylA and xylB. Other promoters for bacterial cells include prokaryotic beta-lactamase (Villa-Kamaroff et al., 1978, Proceedings of the National Academy of Sciences USA 75: 3727-3731), and the tac promoter (DeBoer et al., 1983, Proceedings of the National Academy of Sciences USA 80: 21-25). For an S. cerevisiae host cell, useful promoters include the ENO-1, GAL1, ADH1, ADH2, GAP, TPI, CUP1, PHO5 and PGK promoters. Other useful promoters for yeast host cells are described by Romanos et al., 1992, Yeast 8: 423-488. Still other useful promoters for various host cells are described in “Useful proteins from recombinant bacteria” in Scientific American, 1980, 242: 74-94; and in Sambrook et al., 2001, supra.

A transcription terminator sequence is a sequence recognized by a host cell to terminate transcription, and is typically operably linked to the 3′ terminus of an encoding nucleic acid sequence. Suitable terminator sequences for E. coli host cells include the T7 terminator region. Suitable terminator sequences for yeast host cells such as S. cerevisiae include CYC1, PGK, GAL, ADH, AOX1 and GAPDH. Other useful terminators for yeast host cells are described by Romanos et al., 1992, supra.

A leader sequence is a non-translated region of an mRNA which is important for translation by the host cell. The leader sequence is typically operably linked to the 5′ terminus of a coding nucleic acid sequence. Suitable leaders for yeast host cells include S. cerevisiae ENO-1, PGK, alpha-factor, ADH2/GAP.

A polyadenylation sequence is a sequence operably linked to the 3′ terminus of a coding nucleic acid sequence which, when transcribed, is recognized by the host cell as a signal to add polyadenosine residues to transcribed mRNA. Useful polyadenylation sequences for yeast host cells are described by Guo and Sherman, 1995, Molecular Cellular Biology 15: 5983-5990.

A signal peptide sequence encodes an amino acid sequence linked to the amino terminus of an encoded amino acid sequence, and directs the encoded amino acid sequence into the cell's secretory pathway. In some cases, the 5′ end of the coding nucleic acid sequence may inherently contain a signal peptide coding region naturally linked in translation reading frame, while a foreign signal peptide coding region may be required in other cases. Useful signal peptides for yeast host cells can be obtained from the genes for S. cerevisiae alpha-factor and invertase. Other useful signal peptide coding regions are described by Romanos et al., 1992, supra. An exemplary signal peptide for an E. coli host cell can be obtained from alkaline phosphatase. For a Bacillus host cell, suitable signal peptide sequences can be obtained from alpha-amylase and subtilisin. Further signal peptides are described by Simonen and Palva, 1993, Microbiological Reviews 57: 109-137.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound.

Regulatory systems in prokaryotic systems include the lac, tec, and tip operator systems. For example, one or more promoter sequences can be under the control of an IPTG inducer, initiating expression of the gene once IPTG is added. In yeast, the ADH2 system or GAL1 system may be used. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the respective encoding nucleic acid sequence would be operably linked with the regulatory sequence.

The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. The selectable marker genes can, for example, provide resistance to antibiotics or toxins, complement auxotrophic deficiencies, or supply critical nutrients not in the culture media, and/or provide for control of chromosomal integration. Examples of bacterial selectable markers are the dal genes from Bacillus subtilis or Bacillus licheniformis, or markers which confer antibiotic resistance such as ampicillin, kanamycin, chloramphenicol, or tetracycline resistance. Suitable markers for yeast host cells are ADE2, HIS3, LEU2, LYS2, MET3, TRP1, and URA3.

The vectors of the present invention may also contain one or more elements that permit integration of the vector into the host cell genome or autonomous replication of the vector in the cell independent of the genome. For integration into the host cell genome, the vector may rely on an encoding nucleic acid sequence or other element of the vector for integration into the genome by homologous or nonhomologous recombination. Alternatively, the vector may contain additional nucleotide sequences for directing integration by homologous recombination into the genome of the host cell at a precise location(s) in the chromosome(s).

To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo. Examples of bacterial origins of replication are the origins of replication of plasmids pBR322, pUC19, pACYC177, and pACYC184 permitting replication in E. coli, and pUB1 10, pE194, pTA1060, and pAMβi permitting replication in Bacillus. Examples of origins of replication for use in a yeast host cell are the 2 micron origin of replication, ARS1, ARS4, the combination of ARS1 and CEN3, and the combination of ARS4 and CEN6.

More than one copy of the nucleic acid sequence encoding the L-tryptophane hydroxylase, SPR and a DHPR, and optionally a GCH1, a PTPS, a PCBD1 may be inserted into the host cell to increase production of the gene product. An increase in the copy number of the encoding nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the sequence, can be selected for by cultivating the cells in the presence of the appropriate selectable agent.

### Recombinant Host Cells

The present invention also provides a recombinant host cell, into which a vector according to any preceding embodiment is introduced, typically via transformation, using standard methods known in the art (see, e.g., Sambrook et al., 2001, supra. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizen, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thome, 1987, Journal of Bacteriology 169: 5771-5278).

As described above, the vector, once introduced, may be maintained as a chromosomal integrant or as a self-replicating extra-chromosomal vector.

The transformation can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product, including those referred to above and relating to measurement of 5HTP production. Expression levels can further be optimized to obtain sufficient expression using methods well known in the art and as disclosed herein.

Tryptophan production takes place in all known microorganisms by a single metabolic pathway (Somerville, R. L., Herrmann, R. M., 1983, Amino acids, Biosynthesis and Genetic Regulation, Addison-Wesley Publishing Company, U.S.A.: 301-322 and 351-378; Aida et al., 1986, Bio-technology of amino acid production, progress in industrial microbiology, Vol. 24, Elsevier Science Publishers, Amsterdam: 188-206). The recombinant microbial cell of the invention can thus be prepared from any microbial host cell, using recombinant techniques well known in the art (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999).). Preferably, the host cell is tryptophan autotrophic (i.e., capable of endogenous biosynthesis of L-tryptophan), grows on synthetic medium with suitable carbon sources, and expresses a suitable RNA polymerase (such as, e.g., T7 polymerase).

The microbial host cell for use in the present invention is typically unicellular and can be, for example, a bacterial cell, a yeast host cell, a filamentous fungal cell, or an algeal cell. Examples of suitable host cell genera include, but are not limited to, Acinetobacter, Agrobacterium, Alcaligenes, Anabaena, Aspergillus, Bacillus, Bifidobacterium, Brevibacterium, Candida, Chlorobium, Chromatium, Corynebacteria, Cytophaga, Deinococcus, Enterococcus, Erwinia, Erythrobacter, Escherichia, Flavobacterium, Hansenula, Klebsiella, Lactobacillus, Methanobacterium, Methylobacter, Methylococcus, Methylocystis, Methylomicrobium, Methylomonas, Methylosinus, Mycobacterium, Myxococcus, Pantoea, Phaffia, Pichia, Pseudomonas, Rhodobacter, Rhodococcus, Saccharomyces, Salmonella, Sphingomonas, Streptococcus, Streptomyces, Synechococcus, Synechocystis, Thiobacillus, Trichoderma, Yarrowia and Zymomonas.

In one embodiment, the host cell is bacterial cell, e.g., an Escherichia cell such as an Escherichia coli cell; a Bacillus cell such as a Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or a Bacillus thuringiensis cell; or a Streptomyces cell such as a Streptomyces lividans or Streptomyces murinus cell. In a particular embodiment, the host cell is an E. coli cell. In another particular embodiment, the host cell is of an E. coli strain selected from the group consisting of K12.DH1 (Proc. Natl. Acad. Sci. USA, volume 60, 160 (1968)), JM101, JM103 (Nucleic Acids Research (1981), 9, 309), JA221 (J. Mol. Biol. (1978), 120, 517), HB101 (J. Mol. Biol. (1969), 41, 459) and C600 (Genetics, (1954), 39, 440).

In one embodiment, the host cell is a fungal cell, such as, e.g., a yeast cell. Exemplary yeast cells include Candida, Hansenula, Kluyveromyces, Pichia, Saccharomyces, Schizosaccharomyces and Yarrowia cells. In a particular embodiment, the host cell is an S. cerevisiae cell. In another particular embodiment, the host cell is of an S. cerevisiae strain selected from the group consisting of S. cerevisiae KA31, AH22, AH22R-, NA87-11A, DKD-5D and 20B-12, S. pombe NCYC1913 and NCYC2036 and Pichia pastoris KM71.

### Production of 5HTP

The invention also provides a method of producing 5HTP, comprising culturing the recombinant microbial cell of any preceding aspect or embodiment in a medium comprising a carbon source. 5HTP can then optionally be isolated or retrieved from the medium, and optionally further purified. Importantly, using a recombinant microbial cell according to the invention, the method can be carried out without adding L-tryptophan, THB, or both, to the medium.

Also provided is a method of preparing a composition comprising 5HTP, comprising culturing the recombinant microbial cell of any preceding aspect or embodiment, isolating and purifying 5HTP, and adding any excipients to obtain a composition comprising 5HTP.

Suitable carbon sources include carbohydrates such as monosaccharides, oligosaccharides and polysaccharides. As used herein, “monosaccharide” denotes a single unit of the general chemical formula C_{X}(H₂O)_{y}, without glycosidic connection to other such units, and includes glucose, fructose, xylose, arabinose, galactose and mannose. “Oligosaccharides” are compounds in which monosaccharide units are joined by glycosidic linkages, and include sucrose and lactose. According to the number of units, oligosacchardies are called disaccharides, trisaccharides, tetrasaccharides, pentasaccharides etc. The borderline with polysaccharides cannot be drawn strictly; however the term “oligosaccharide” is commonly used to refer to a defined structure as opposed to a polymer of unspecified length or a homologous mixture. “Polysaccharides” is the name given to a macromolecule consisting of a large number of monosaccharide residues joined to each other by glycosidic linkages, and includes starch, lignocellulose, cellulose, hemicellulose, glycogen, xylan, glucuronoxylan, arabinoxylan, arabinogalactan, glucomannan, xyloglucan, and galactomannan. Other suitable carbon sources include acetate, glycerol, pyruvate and gluconate. In one embodiment, the carbon source is selected from the group consisting of glucose, fructose, sucrose, xylose, mannose, galactose, rhamnose, arabinose, fatty acids, glycerine, glycerol, acetate, pyruvate, gluconate, starch, glycogen, amylopectin, amylose, cellulose, cellulose acetate, cellulose nitrate, hemicellulose, xylan, glucuronoxylan, arabinoxylan, glucomannan, xyloglucan, lignin, and lignocellulose. In one embodiment, the carbon source comprises one or more of lignocellulose and glycerol.

The culture conditions are adapted to the recombinant microbial host cell, and can be optimized to maximize 5HTP production by varying culture conditions and media components as is well-known in the art.

For a recombinant Escherichia coli cell, exemplary media include LB medium and M9 medium (Miller, Journal of Experiments in Molecular Genetics, 431-433, Cold Spring Harbor Laboratory, New York, 1972), optionally supplemented with one or more amino acids. When an inducible promoter is used, the inductor can also be added to the medium. Examples include the lac promoter, which can be activated by adding isopropyl-beta-thiogalactopyranoside (IPTG) and the GAL promoter, in which case galactose can be added. The culturing can be carried out a temperature of about 10 to 50° C. for about 3 to 72 hours, if desired, with aeration or stirring.

For a recombinant Bacillus cell, culturing can be carried out in a known medium at about 30 to 40° C. for about 6 to 40 hours, if desired with aeration and stirring. With regard to the medium, known ones may be used. For example, pre-culture can be carried out in an LB medium and then the main culture using an NU medium.

For a recombinant yeast cell, Burkholder minimum medium (Bostian, K. L., et al. Proc. Natl. Acad. Sci. USA, volume 77, 4505 (1980)) and SD medium containing 0.5% of Casamino acid (Bitter, G. A., et al., Proc. Natl. Acad. Sci. USA, volume 81, 5330 (1984) can be used. The pH is preferably adjusted to about 5-8. Culturing is preferably carried out at about 20 to about 40° C., for about 24 to 84 hours, if desired with aeration or stirring.

In one embodiment, the method for producing 5HTP further comprises adding THB exogenously to the culture medium, optionally at a concentration of 0.01 to 100 mM, such as a concentration of 0.05 to 10 mM, such as about 0.1 mM or 1 mM. This may be done, for example, when the recombinant host cell has been transformed with the second (regenerating) THB pathway but not the first THB pathway. In another embodiment, both L-tryptophan and THB are added exogenously, with L-tryptophan at a concentration of 0.01 to 10 g/L, optionally 0.1 to 5 g/L, such as 0.2 to 1.0 g/L. In one embodiment, no L-tryptophan is added. In another embodiment, no L-tryptophan or THB is added to the medium, so that the 5HTP production relies on endogenously biosynthesized substrates.

Using the method for producing 5HTP according to the invention, a 5HTP yield of at least about 0.5%, such as at least about 1%, at least about 5%, at least 10%, at least 20%, at least 30%, at least 40%, at least 50%, at least 60%, at least 70%, at least 80% or at least 90% of the theoretically possible yield can be obtained from a suitable carbon source, such as glucose. In one embodiment, the method achieves a yield of at least about 1% from a medium comprising glucose, in the absence of added THB and/or L-tryptophan to the medium.

Isolation of 5HTP from the cell culture can be achieved, e.g., by separating the 5HTP from the cells using a membrane, using, for example, centrifugation or filtration methods. The 5-HTP-containing supernatant is then collected. Further purification of the 5HTP can then be carried out using known methods, such as, e.g., salting out and solvent precipitation; molecular-weight-based separation methods such as dialysis, ultrafiltration, and gel filtration; charge-based separation methods such as ion-exchange chromatography; and methods based on differences in hydrophobicity, such as reversed-phase HPLC; and the like. In one embodiment, ion-exchange chromatography is used to purify the 5HTP. An exemplary method for 5HTP purification using ion-exchange chromatography is described in Bakri and Carlsson (Anal Biochem 1970; 34:46-65).

Once a sufficiently pure 5HTP preparation has been achieved, suitable excipients, stabilizers can optionally be added and the resulting preparation incorporated in a composition for use in preparing a product such as, e.g., a dietary supplement, a pharmaceutical, a cosmeceutical, or a nutraceutical. For a dietary supplement, each serving can contain, e.g., from about 1 mg to about 900 mg 5HTP, such as from about 20 mg to about 200 mg, or about 100 mg. Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives may also be added to the nutritional supplement to extend product shelf life.

Preferably, preservatives such as potassium sorbate, sodium sorbate, potassium benzoate, sodium benzoate or calcium disodium EDTA are used.

### Example 1

### A Metabolic Pathway for Producing 5-Hydroxy-L-Tryptophan from L-Tryptophan in a Microorganism

This example describes the introduction of a pathway for producing 5-Hydroxy-L-tryptophan from L-tryptophan, into E. coli. 5-Hydroxy-L-tryptophan is derived from the native metabolite L-tryptophan in one enzymatic step as shown in FIG. 1. The enzyme that catalyzes this reaction is tryptophan hydroxylase (TPH1, EC 1.14.16.4), which requires both oxygen and Tetrahydropterin (THB) as cofactors. Specifically, the enzyme catalyzes the conversion of L-tryptophan and THB into 5-Hydroxy-L-tryptophan and 4a-hydroxytetrahydrobiopterin (HTHB). In the following examples, for the production of 5-Hydroxy-L-tryptophan from L-tryptophan, we used TPH genes from variant organisms such as, a double truncated TPH1 from Oryctolagus cuniculus (rabbit) having the sequence of SEQ ID NO:1 (encoded by SEQ ID NO:40), TPH2 from Homo sapiens having the sequence of SEQ ID NO:2, and TPH1 from Gallus gallus having the sequence of SEQ ID NO:6. The rationale for using the truncated form rather than the wild-type enzyme was to increase the heterologous expression and stability of the enzyme by removing both the regulatory and interface domains (Moran, Daubner, & Fitzpatrick, 1998). In addition, this mutant enzyme has been shown to be soluble in E. coli, and have high specific activity.

THB is not native to E. coli, so any THB production capability needs to be added to the bacteria. A previous study reported the production of THB in E. coli from the native metabolite Guanosine triphosphate (GTP) in a 3-enzymatic process (Yamamoto, 2003). For the synthesis of THB, the first enzymatic step is GTP cyclohydrolase I (GCH1, EC 3.5.4.16), which catalyzes the conversion of GTP and water into 7,8-dihydroneopterin 3′-triphosphate and formate. For the following examples, a GCHI that is native to E. coli (encoded by SEQ ID NO:41) is used, which has many aspects of its enzymatic kinetics and reaction mechanisms uncovered (NARP et al., 1995) (Schramek et al., 2002) (Schramek et al., 2001) (Rebelo et al., 2003). The second reaction in the production of THB from GTP is a 6-pyruvoyl-tetrahydropterin synthase (PTPS, EC 4.2.3.12), which catalyzes the synthesis of 7,8-dihydroneopterin 3′-triphosphate (DHP) into 6-pyruvoyltetrahydropterin (6PTH) and triphosphate (FIG. 1). For the following examples, a PTPS from Rattus norvegicus (Rat) is used (encoded by SEQ ID NO:42), which was used in the Yamamoto (2003) study mentioned above to produce THB from GTP in E. coli. The final reaction in the production of THB from GTP, is the conversion of 6PTH into THB, via NADPH oxidation (FIG. 1), and is carried out by the NADPH-dependent Sepiapterin reductase (SPR, EC:1.1.1.153). Similar to the PTPS enzyme above, for this example, an SPR from Rat is used (encoded by SEQ ID NO:43), which was also used in a previous study to produce THB from GTP in E. coli (Yamamoto, 2003).

As mentioned above, when producing 5-Hydroxy-L-Tryptophan from L-Tryptophan using a TPH1, THB is converted to HTHB. Due to the high price of THB, addition to the media is not cost-efficient, thus HTHB must be converted back to THB, and for the following examples, a 2-step enzymatic process is used. The first enzymatic step is 4a-hydroxytetrahydrobiopterin dehydratase (PCBD1, EC: 4.2.1.96), which catalyzes the conversion of HTHB into Dihydrobiopterin (DHB) and water. A PCBD1 from Pseudomonas aeruginosa is used (SEQ ID NO:44), which has been previously expressed in E. coli, and purified for characterized (Köster et al., 1998). The second enzymatic step is a NADH-dependent dihydropteridine reductase (DHPR, EC: 1.5.1.34), which catalyzes the conversion of DHB into THB, via the oxidation of NADH. For this example, a DHPR that is native to E. coli (SEQ ID NO:45) is used (Vasudevan et al., 1988).

### Example 2

### Construction of DNA Constructs for Producing 5-Hydroxy-L-Tryptophan from L-Tryptophan in a Microorganism

Methods have recently been developed for assembling of multiple overlapping DNA molecules (Gibson et al., 2008) (Gibson et al., 2009) (Li & Elledge, 2007). One of these methods allows the assembly multiple overlapping DNA fragments by the concerted action of an exonuclease, a DNA polymerase and a DNA ligase. The DNA fragments are first recessed using an exonuclease; yielding single-stranded DNA overhangs that can be specifically annealed. This assembly is then covalently joined using a DNA polymerase and DNA ligase. This method was used to assemble DNA molecules the complete synthetic 583 kb genitalium genome, and has also produced products as large as 900 kb. For the production of 5-Hydroxy-L-tryptophan from L-tryptophan, we used this method to generate a 12,737 bp BAC that contains the enzymes GCH1, PTPS, SPR, TPH1, DHPR, and PCBD1, all under the control of T7 promoter or lac promoter or a constitutive promoter.

A DNA operon for the production of THB from GTP was synthesized containing SEQ ID NOS:41, 42 and 43 under control of the T7 promoter region (SEQ ID NO:46) or lac promoter region (SEQ ID NO:62) and T7 terminator region (SEQ ID NO:47). In order for strong translation, genes within an operon were separated by an 18 bp intragenic region, which contained an optimized ribosomal binding site (SEQ ID NO:48). Furthermore, a linker region 1 (SEQ ID NO:49) was added upstream of the T7 or lac RNA polymerase promoter site, which had homology to the last ˜200 bases on the 3′ end of PCR amplified pCC1BAC. A linker region 2 (SEQ ID NO:50) was added downstream of the T7 RNA polymerase terminator site, and had homology to the last ˜200 bases on the 5′ end TRP operon described below. Furthermore, the Linker regions had NotI restriction digest sites on the ends, and the entire construct was cloned into the plasmid. Thus, a final construct pTHB (SEQ ID NO:51) was generated, which contained the following sequences, and in the following order: SEQ ID NO:49, 46, 41, 48, 42, 48, 43, 47, 50. In order to release the operon for the anneal/repair reaction below, 500 ug of pTHB was digested, purified of salts using ethanol precipitation, and then stored at −20 C.

A second DNA operon was synthesized for the production of 5-Hydroxy-L-tryptophan from L-tryptophan, in addition to regeneration of THB from HTHB. This operon contained SEQ ID NOS:40, 44 and 45 under control of the T7 promoter region (SEQ ID NO:46), or the lac promoter region (SEQ ID NO:62), and T7 terminator region (SEQ ID NO:47). In order for strong translation, genes within an operon were separated by an 18 bp intragenic region, which contained an optimized ribosomal binding site (SEQ ID NO:48). A linker region 2 (SEQ ID NO:50) was added upstream of the T7 RNA polymerase promoter site, which is the same linker added to the plasmid pTHB, to assist in the assembly of the final plasmid. The DNA construct was cloned into the standard cloning vector pUC57 with flanking NotI restriction digestion sites, thus allowing extraction of DNA construct when necessary. The final construct pTRP (SEQ ID NO:52) was generated, which contained the following sequences, and in the following order: SEQ ID NO:49, 46, 40, 48, 44, 48, 45, 47, 50. As in the case with pTHB, in order to release the operon for the anneal/repair reaction below, 500 ug of pTRP was digested, purified of salts using ethanol precipitation, and then stored at −20° C.

In order to generate the BAC backbone for the final DNA construct, pCC1BAC (EPICENTRE) was PCR-amplified using primer A (SEQ ID NO:53), and primer B (SEQ ID NO:54), and then gel purified. Assembly reactions (80 μl) were carried out in 250 μl PCR tubes in a thermocycler and contained 5% PEG-8000, 200 mM Tris-Cl pH 7.5, 10 mM MgCl2, 1 mM DTT, 100 μg/ml BSA, and 4.8 U of T4 polymerase. All DNA pieces in the assembly reaction must be at equal Molar concentrations. Thus, 500 ng of digested plasmids pTHB and pTRP, were added to the reaction, in addition to 1000 ng of the pCC1BAC PCR product using primers A and B. Reactions were incubated at 37° C. for a period of 10 minutes. The reactions were then incubated at 75° C. for 20 minutes, cooled at −6° C./minute to 60° C. and then incubated for 30 minutes. Following the 30-minute incubation, the reaction was cooled at −6° C./min to 4° C. and then held. The assembly reaction was followed by a repair reaction, which repairs the nicks in the DNA. The repair reaction, which was a total of 40 μl, contained 10 μl of the assembly reaction, 40 U Taq DNA ligase, 1.2 U Taq DNA Polymerase, 5% PEG-8000, 50 mM Tris-CI pH 7.5, 10 mM MgCl₂, 10 mM DTT, 25 μg/ml BSA, 200 μM each dNTP, and 1 mM NAD. The reaction was incubated for 15 min at 45° C., and then stored at −20° C.

A similar approach was applied for the constructions of DNA vectors for the expression of TPH genes from Oryctolagus cuniculus (SEQ ID NO:1, encoded by SEQ ID NO:40), Homo sapiens (SEQ ID NO:2) or Gallus gallus (SEQ ID NO:6). A linear DNA was amplified by PCR using cloning vectors pBAD18kan as a template using primers Lin-pBAD-FWD (SEQ ID NO:64) and Lin-pBAD-REV (SEQ ID NO:65). The TPH genes were amplified using the primers TPH-FWD (SEQ ID NO:66) and TPH-REV (SEQ ID NO:67). The PCR amplified DNA fragments were assembled using the above mentioned approach.

A similar approach was applied for the construction of DNA vector for the expression of GCH1, PTPS and SPR genes (SEQ ID NOS:41, 42 and 43) for the synthesis of THB. A DNA operon for the production of THB from GTP was amplified using primers THB-FWD (SEQ ID NO:76) and THB-REV (SEQ ID NO:77) using p5HTP as the template, and the vector backbone was amplified using pTH19cr (SEQ ID NO:78) as the template using primers pTH19cr-Lin-FWD (SEQ ID NO:79) and pTH19cr-Lin-REV (SEQ ID NO:80). The PCR fragments were assembled using the above mentioned approach, and the final constructed plasmid was designated pTHB (SEQ ID NO:93, FIG. 6), where the THB synthetic pathway genes are under the control of lac promoter.

A similar approach was applied for the construction of DNA vector for the expression of PCBD1 and DHPR genes (SEQ ID NO:29 and 34, respectively). The genes were PCR amplified using primers DP-FWD (SEQ ID NO:81) and DP-REV (SEQ ID NO:82) using p5HTP as the template. The vector backbone was PCR amplified using pUC18 (SEQ ID NO:83) as the template using primers LinPUC18-FWD (SEQ ID NO:84) and LinPUC18-REV (SEQ ID NO:85). The linearized PCR products were assembled using the above-described approaches, and the final constructed plasmid was designated pDP, where the PCBD1 and DHPR genes are under the control of lac promoter.

A similar approach was applied for the constructions of DNA vectors for the expression of the GCH1, PTPS, SPR, TPH1 genes and the PCBD1 and DHPR genes. The operon containing the lac promoter, PCBD1 and DHPR genes was PCR amplified using the pDP as the template and using the primers lac-DP-FWD (SEQ ID NO:86) and lac-DP-REV (SEQ ID NO:87). The operon containing the lac promoter, GCH1, PTPS, SPR, TPH1 genes was PCR amplified using the pTHB as the template and using primers Pa-THB-FWD (SEQ ID NO:89) and Pa-THB-REV (SEQ ID NO:90). The vector backbone was amplified using pBAD33 (SEQ ID NO:91) as the template and primers Lin-pBAD-FWD (SEQ ID NO:64) and Lin-pBAD-REV (SEQ ID NO:65).

The amplified linear DNA fragments were assembled using the above mentioned protocol, and the final constructed plasmid was designated pTHBDP (SEQ ID NO:92, FIG. 5).

### Example 3

### Transformation of E. coli Cells with DNA Constructs for Producing 5-Hydroxy-L-Tryptophan from L-Tryptophan in a Microorganism

In a 2 mm cuvette, five microliters of the repair reaction was electroporated into 50 uL of EPI300 E. coli cells (EPICENTRE) using a MicroPulser Electroporator (BioRad). Directly following the electroporation, cells were transferred to 500 uL SOC media (2% peptone, 0.5% Yeast extract, 10 mM NaCl, 2.5 mM KC, 10 mM MgCl2, 10 mM MgSO4, 20 mM Glucose) and incubated at 37° C. for 2 hours. Cells were then plated onto LB agar supplemented with 15 μg/m chloramphenicol or 50 μg/ml kanamycine depending on the vector backbone sequence, and incubated overnight at 37° C. Yields typically depend on the size of overlapping regions, the size of the final construct, and the number of DNA pieces that are being assembles. Specifically, shorter overlapping regions, larger final constructs, and higher number of assembly pieces all lead to a decrease in yields. In this assembly, there were 3 DNA pieces being assembled with ˜60-200 bp overlapping regions. It is best to keep the overlapping regions 200 bps or more, however, 60 pbs is sufficient but leads to low yields. In addition, the final construct was only 12,737 bps, which is relatively small for this methodology, and thus has little effect on the efficiency and yields. The following day, 10 colonies are selected, and grown overnight in LB medium (1% peptone, 0.5% yeast extract, and 0.5% NaCl) supplemented with 15 μg/m chloramphenicol or 50 μg/ml of kanamycin depending on the vector backbone sequence. DNA is extracted from each overnight culture using a GeneJET Plasmid Miniprep Kit (Fermentas).

BAC DNA constructs were digested with the restriction enzyme SalI (NEB) and subjected to agarose gel electrophoresis using mini sub cell (Bio-Rad) for 30 minutes at 100V. A 7006 bp band (pCC1BAC) and 5731 bp band (THB-TRP fragment) were observed, ensuring the correct assembly of the DNA construct. In order to confirm correct assembly, ˜500 bp regions surround the overlapping regions were PCR amplified. The overlapping region of pCC1BAC and THB operon was amplified with primers C (SEQ ID NO:55) and D (SEQ ID NO:56), the assembly region of the THB and TRP operon was amplified with primers E (SEQ ID NO:57) and F (SEQ ID NO:58), and the assembly region of the TRP operon and pCC1BAC was amplified using primers G (SEQ ID NO:59) and H (SEQ ID NO:60). The final DNA construct for producing 5-Hydroxy-L-tryptophan from L-tryptophan in a microorganism was thus confirmed and designated p5HTP (FIG. 2) (SEQ ID NO:61).

DNA constructs based on pBAD18kan extracted from overnight culture were digested with BamHI and subjected to agarose gel electrophoresis. The clones with expected band sizes were sequenced and confirmed. The plasmid harboring TPH2 from Homo sapiens was designated pTPH-H (SEQ ID NO:68), the plasmid harboring TPH1 from Gallus gallus was designated pTPH-G (SEQ ID NO:69), and the plasmid harboring TPH1 from Oryctolagus cuniculus was designated pTPH_OC (SEQ ID NO:70).

### Example 4

### Transformation of T7 RNA Polymerase Harboring Cells with p5HTP, and Fermentation for the Production of 5-Hydroxy-L-Tryptophan from L-Tryptophan in a Microorganism

The p5HTP DNA construct was then introduced into an E. coli host cell harboring the T7 RNA polymerase. The strain chosen was the Origami B (DE3) (EMD Chemicals), which contains a T7 RNA polymerase under the control of an IPTG inducer. Origami B (DE3) strains also harbor a deletion of the lactose permease (lacY) gene, which allows uniform entry of IPTG into all cells of the population. This produces a concentration-dependent, homogeneous level of induction, and enables adjustable levels of protein expression throughout all cells in a culture. By adjusting the concentration of IPTG, expression can be regulated from very low levels up to the robust, fully induced levels commonly associated with T7 RNA polymerase expression. In addition, Origami B(DE3) strains have also been shown to yield 10-fold more active protein than in another host even though overall expression levels were similar.

Origami B(DE3) strains containing p5HTP were evaluated for the ability to produce 5HTP. Given that an industrial process would require the production of chemicals from low-cost carbohydrate feedstocks such as glucose, it is necessary to demonstrate the production of 5HTP from a native compound in E. coli. In this example, L-Tryptophan was used as the starting metabolic intermediate compound, and the metabolic pathways for the production of L-Tryptophan are native to E. coli, and well-known. Thus, the next set of experiments was aimed to determine whether endogenous L-tryptophan produced by the cells during growth on glucose could fuel the 5HTP pathway. Cells were grown aerobically in M9 minimal medium (6.78 g/L, Na₂HPO₄, 3.0 g/L KH₂PO₄, 0.5 g/L NaCl, 1.0 g/L NH₄Cl, 1 mM MgSO₄, 0.1 mM CaCl₂) supplemented with 10 g/L glucose, 1 g/L L-tryptophan, 100 mM 3-(N-morpholino)propanesulfonic acid (MOPS) to improve the buffering capacity, and the 15 mg/L chloramphenicol. In order to determine the optimal Induction level, growth experiments were done with IPTG concentrations of 1000, 100, and 10 μM. IPTG was added when the cultures reached an OD600 of approximately 0.2, and samples were taken for 5HTP analysis at 12 hours following induction. Significant amounts of 5HTP were detected at all IPTG concentrations, indicating that the basal level of expression was quite high. Maximum 5HTP concentrations of almost 1 mg/L were achieved when using 1 mM IPTG induction.

### Example 5

### Knocking-Out tnaA Gene in E. coli to Prevent 5-Hydroxytryptophan Degradation

This Example shows that tryptophanase, apart from degrading tryptophane to indole, can also degrade 5-hydroxytryptophan to 5-hydroxyindole (FIG. 3):

E. coli MG1655 wild type strain was streaked out on a LB culture plate. After incubating overnight at 37° C., a single colony was picked for the inoculation of 5 ml of LB medium supplemented with 1.0 mM of 5-hydroxytryptophan in a 14 ml falcon tube, and the cultures were incubated at 37° C. with a shaking speed of 250 rpm. After 24 hours, a significant portion of 5-hydroxytryptophan was degraded into 5-hydroxyindole, and after 96 hours, all the 5-hydroxytryptophan was degraded (FIG. 4a).

We knocked out the tnaA gene using the Datsenko-Wanner method (Datsenko and Wanner 2000). A replacement DNA fragment was PCR amplified using the primers H1-P1-tnaA (SEQ ID NO:71) and H2-P2-tnaA (SEQ ID NO:72), and pKD4 as template as indicated in the referenced article. The PCR product was digested with DpnI, and then purified. As indicated by the referenced article, the purified DNA product for gene knockout was transformed into E. coli MG1655 competent cell carrying a helper plasmid pKD46 expresses λ-red recombinase. The transformants were spread out on kanamycin LB culture plates, and leave at 30° C. overnight. The colonies that grew up on kanamycin plates were restreaked on fresh LB plates containing kanamycin, and the isolated colonies were checked by colony PCR with primers tnaA-CFM-FWD (SEQ ID NO:73) and K1 (SEQ ID NO:75) to confirm gene knockout.

The confirmed knockout strain E. coli MG1655 tnaA::FRT-Kan-FRT was cultured in LB medium supplemented with 50 μg/ml of kanamycin, and then washed with cold glycerol to prepare competent cells. Then another helper plasmid pCP20 was transformed into the knockout strain and the transformants were spread out on LB culture plates with ampicillin as selection marker. The plates were kept at 30° C. till colonies grow up on it. Selected single colonies were grown in LB medium supplemented with ampicillin overnight at 30° C. Cell pellets were collected by centrifugation and washed twice with fresh LB medium. Then the cell pellets were resuspended in LB medium and cultured at 37° C. for 3 hours so that it may lose the helper plasmid pCP20. After that the cell pellets were collected, washed, and then spread out on LB plates. After incubating at 37° C. overnight, single colonies were restreaked out on LB, LB plus kanamycin, and LB plus ampicillin plates. The colonies that grew on LB plates, but not on LB plus kanamycin or LB plus ampicillin plates, were selected for colony PCR confirmation with tnaA-CFM-FWD (SEQ ID NO:73) and tnaA-CFM-REV (SEQ ID NO:74).

The confirmed E. coli MG1655 tnaA⁻ mutant strain was then tested. The strain was inoculated in LB medium supplemented with 1.0 mM of 5-hydroxytryptophan, and then incubated at 37° C. with a shaking speed of 250 rpm. As a control, E. coli MG1655 wild type strain was cultured under the same condition. Samples were taken after 48 hours. The results showed that the 5-hydroxytryptophan was completed degraded into 5-hydroxyindole in the culture of wild type strain, while 5-hydroxytryptophan was stable in the culture of tnaA⁻ mutant strain (FIG. 4b).

### Example 6

### Transformation of E. coli MG1655 tnaA⁻ Mutant Cell with pTPH-H or pTPH-G Together with pTHBDP, and Fermentation for the Production of 5-Hydroxy-L-Tryptophan

The constructed pTPH-H, pTPH_OC or pTPH-G were co-transformed with pTHBDP into E. coli MG1655 tnaA⁻ mutant strain, and the cells were tested for 5-hydroxy-L-tryptophan production in shake flask cultures.

### Cell Culture Conditions.

A single colony of the E. coli MG1655 tnaA⁻ mutant strain carrying the plasmids pTHBDP and pTPH-H or pTPH-G was used for the inoculation of 5 ml LB medium with 15 μg/ml of chloramphenicol and 50 μg/ml of kanamycin. The culture was incubated in a shaker at 30° C. and a rotation speed at 250 rpm. The cell pellets were collected at exponential phase by centrifugation, washed twice with fresh LB medium, and then resuspended in 50 ml of LB medium supplemented with 5 g/L of glycerol and 0.2 g/L of tryptophan. The culture mediums were prepared separately, and 100 μl of resuspended preculture cell solution was used for the inoculation of 5 ml fresh culture medium. The culture tubes were incubated in a shaker at 37° C. and a rotation speed at 200 rpm. After the cultures grow to OD600 about 0.5, 0.1 mM of IPTG was added to induce protein expression. Culture broth was collected 24 hours after induction and centrifuged at 8000 rpm for 5 min. Supernatants were collected for HPLC measurements.

HPLC Conditions.

A Ultimate 3000 HPLC system (Dionex, now Thermo-fisher) was used for this assay. The mobile phase of the HPLC measurement was 80% 10 mM NH₄COOH adjusted to pH 3.0 with HCOOH and 20% acetonitrile. The flow rate was set at 1.0 ml/min. A Discovery HS F5 column (Sigma) was used for the separation, and an UV detection at 254 nm was used for 5-hydroxytryptophan detection. The column temperature was set at 35° C. The standard 5-hydroxytryptophan (Sigma, >98% purity) was used to establish a standard curve for 5HTP concentrations.

### Results

Using tnaA⁻ cells, the 5-hydroxytryptophan concentrations measured in the cultures ranged from 0.15 mM to 0.9 mM. The highest production was observed with cells harboring plasmid expressing TPH1 from Oryctolagus cuniculus, producing 0.9 mM of 5-hydroxy-L-tryptophan in the cultures.

Table 1 shows the results of a preliminary experiment using E. coli MG1655 cells (without tnaA knock-out) transformed with pTPH-H. Since the analytical method used was not at the time fine-tuned, the results were interpreted as qualitative rather than quantitative. The data showed, however, that adding THB did not help 5HTP production, and that the pathway for 5HTP production was functional.

TABLE 1
Summarized HPLC Data

| Culture code | Medium | 5HTP (mM) |
|---|---|---|
| A | M9 + 10 g/L Glc + 1.0 g/L Trp + MOPS | 0.66 |
| B | M9 + 5 g/L Glc | 0.28 |
| C | M9 + 5 g/L Glc + 0.2 g/L Trp | 0.42 |
| D | M9 + 5 g/L Glc + 1 mM THB | 0.13 |
| E | M9 + 5 g/L Glc + 0.2 g/L Trp + 1 mM THB | 0.39 |
| F | LB + 0.2 g/L Trp | 1.45 |
| G | LB + 5 g/L Glc + 0.2 g/L Trp | 1.42 |
| H | LB + 0.2 g/L Trp + 1 mM THB | 1.24 |
| I | LB + 5 g/L Glc + 0.2 g/L Trp + 1 mM THB | 1.89 |
| J | LB + 5 g/L Glc | 2.44 |
| K | LB + 5 g/L Glc + 1 mM THB | 1.51 |
| M9 | M9 + 5 g/L Glc | 0.12 |
| MG1655 | LB + 5 g/L Glc | 0.02 |

### Example 7

### Constructing 5-Hydroxytryptophan Producer in Saccharomyces cerevisiae

Saccharomyces cerevisiae strains do not have native tryptophan hydroxylase or THB synthesis- or recycling pathways. These genes/pathways must be cloned into the S. cerevisiae strain in order to produce 5-hydroxytryptophan. Mikkelsen et al. (2012) has introduced a platform for chromosome integration and gene expression in S. cerevisiae strains, which can be used for the construction of 5-hydroxytryptophan producers.

The THB synthetic pathway genes are assigned to be expressed at relatively low levels, and therefore the X3 and X4 sites (Mikkelsen et al., 2012) are chosen for the expression of the GCH1, PTPS and SPR genes (SEQ ID NOS:41, 42, and 43). These three genes can be PCR amplified with using pTHB plasmid (SEQ ID NO:93, FIG. 6) as the template and primers GCH1-FWD, GCH1-REV, PTPS-FWD, PTPS-REV, SPR-FWD, and SPR-REV, respectively (SEQ ID NOS:94-99, respectively). The amplified PCR products are fused into the X3 and X4 vectors together with the bidirectional promoter fragment (Mikkelsen et al., 2012) using the USER cloning protocol (Nour-Eldin et al. 2006).

A similar approach can be used for the construction of the insertion vectors for the THB recycling pathway genes such as DHPR and PCBD1 (SEQ ID NOS:34 and 29, respectively). The DHPR and PCBD1 genes can be amplified using the primers DHPR-FWD, DHPR-REV, PCBD1-FWD, and PCBD1-REV, respectively (SEQ ID NOS:100-103). The insertion vector XI-4 is chosen as the backbone (Mikkelsen et al. 2012).

A similar approach can be used for the construction of the insertion vectors for the expression of TPH2 gene from Homo sapiens (SEQ ID NO:2), TPH1 from Gallus gallus (SEQ ID NO:6), and TPH1 gene from Oryctolagus cuniculus (SEQ ID NO:1). The primers used for the amplification of these genes are TPH-H-FWD, TPH-H-REV, TPH-G-FWD, TPH-G-REV, TPH-Oc-FWD, and TPH-OC-REV, respectively (SEQ ID NOs:104-109). The XI-3 insertion vector is used for the construction (Mikkelsen et al. 2012).

Transformation of the above mentioned insertion plasmids is achieved using the lithium acetate/single-stranded carrier DNA/PEG method (Gietz and Schiestl, 2007). The above-described insertion plasmids for the integration of THB synthesis and recycling pathway genes are transformed iteratively into the yeast strain CEN.PK113-7D in three consecutive transformations. The URA3 marker is eliminated by direct repeat recombination after each integration by selecting colonies growing on plates with 740 mg/L 5-fluoroorotic acid. The colonies grown up on the selection plates are further screened by colony PCR to confirm the insertions. The selected strain(s) are used to prepare competent cells, which are then transformed with one of the TPH insertion plasmids as described above. The transformant mixtures are screened with uracil and 5-fluoroorotic acid, and further confirmed with colony PCR. The final strains are named as CEN.PK-TPHh, CEN.PK-TPHg, and CEN.PK-TPHoc carrying and expressing the TPH genes from Homo sapiens, Gallus gallus, and Oryctolagus cuniculus, respectively.

### LIST OF REFERENCES

Datsenko, K. A. and B. L. Wanner (2000). One-step inactivation of chromosomal genes in Escherichia coli K-12 using PCR products. Proceedings of the National Academy of Sciences 97(12): 6640-6645.
Gibson, D. G., et al. (2008). Complete Chemical Synthesis, Assembly, and Cloning of a Mycoplasma genitalium Genome. Science, 319, 1215-1220.
Gibson, D. G., et al. (2009). Enzymatic assembly of DNA molecules up to several hundred kilobases. Nature Methods, 6 (5), 343-345.
Gietz, R. D. and R. H. Schiestl (2007). Large-scale high-efficiency yeast transformation using the LiAc/SS carrier DNA/PEG method. Nature Protocols 2(1): 38-41.
Katsuhiko Y, et al. Genetic engineering of Escherichia coli for production of tetrahydrobiopterin. Metabolic engineering, vol. 5(4), 246-54.
Köster, S., et al. (1998). Pterin-4a-Carbinolamine Dehydratase from Pseudomonas aeruginosa: Characterization, Catalytic Mechanism and Comparison to the Human Enzyme. 379, 1427-1432.
Li, M. Z., Elledge, S. J. (2007). Harnessing homologous recombination in vitro to generate recombinant DNA via SLIC. Nature Methods, 4 (3), 251-256.
McKinney J., et al. (2004). Expression and purification of human tryptophan hydroxylase form Eschericia coli and Pichia pastoris. Protein Expression and Purification, 33(2), 185-194.
Mikkelsen M. D. et al., (2012) Microbial production of indolylgclucosinolate through engineering of a multi-gene pathway in a versatile yeast expression platform. Metab. Eng. 14: 104-111.
Moran, R. G., Daubner, C. S., & Fitzpatrick, P. F. (1998). Expression and Characterization of the Catalytic Core of Tryptophan Hydroxylase. Journal of Biological Chemistry, 273 (20), 12259-12266.
Narp, H., et al. (1995). Active site topology and reaction mechanism of GTP cyclohydrolase I. Proc. Natl. Acad. Sci. USA, 92, 12120-12125.
Nour-Eldin H H et al. (2006) Advancing uracil-excision based cloning towards an ideal technique for cloning PCR fragments. Nucleic Acids Res. 34(18):e122
Rebelo, J., et al. (2003). Biosynthesis of Pteridines. Reaction Mechanism of GTP Cyclohydrolase I. J. Mol. Biol., 326c, 503-516.
Schoedon, G., et al. (1992). Allosteric characteristics of GTP cyclohydrolase I from Escherichia coli. Eur. J. Biochem, 210, 561-568.
Schramek, N., et al. (2002). Reaction Mechanism of GTP Cyclohydrolase I Single Turnover Experiments Using a Kinetically Competent Reaction Intermediate. J. Mol. Biol., 316, 829-837.
Schramek, N., et al. (2001). Ring Opening Is Not Rate-limiting in the GTP Cyclohydrolase I Reaction. Journal of Biological Chemistry, 276 (4), 2622-2626.
Vasudevan, S. G., et al. (1988). Dihydropteridine reductase from Escherichia coli*. Biochem. J., 255, 581-588.
Winge et al. (2008), Biochem 3 410:195-204.
Watanabe T and Snell E E (1977). The interaction of Escherichia coli tryptophanase with various amino and their analogs. Active site mapping. J Biochem 82(3); 733-45.
Windahl M. S., et al. Expression, purification and enzymatic characterization of the catalytic domains of human tryptophan hydroxylase isoforms. J Protein Chem 28(9-10), 400-406.
Yamamoto, K. (2003). Genetic engineering of Escherichia coli for production of tetrahydrobiopterin. Metabolic Engineering, 5, 246-254.
U.S. Pat. No. 3,830,696
U.S. Pat. No. 3,808,101
U.S. Pat. No. 7,807,421 B2
U.S. Pat. No. 6,180,373 B1
U.S. 2001/0049126

Throughout this application, various publications have been referenced. The disclosure of each one of these publications in its entirety is hereby incorporated by reference in this application in order to more fully describe the state of the art to which this invention pertains. Although the invention has been described with reference to the Examples provided above, it should be understood that various modifications can be made without departing from the spirit of the invention.

### Embodiments

The following represent specific, exemplary embodiments of the present invention.

1. A recombinant microbial cell comprising an exogenous nucleic acid sequence encoding an L-tryptophan hydroxylase (TPH) (EC 1.14.16.4), and exogenous nucleic acids encoding enzymes of at least one pathway for producing tetrahydrobiopterin (THB).

2. The recombinant microbial cell of embodiment 1, comprising exogenous nucleic acids encoding enzymes of a first and/or a second pathway for producing THB, the first pathway producing THB from guanosin triphosphate (GTP), and the second pathway regenerating THB from 4a-hydroxytetrahydrobiopterin.

3. The recombinant microbial cell of any one of the preceding embodiments, comprising exogenous nucleic acids encoding
(a) optionally, a GTP cyclohydrolase I (EC 3.5.4.16);
(b) a 6-pyruvoyl-tetrahydropterin synthase (EC 4.2.3.12); and
(c) a sepiapterin reductase (EC 1.1.1.153).

4. The recombinant microbial cell of any one of the preceding embodiments, comprising exogenous nucleic acids encoding
(a) a 4a-hydroxytetrahydrobiopterin dehydratase (EC 4.2.1.96); and
(b) optionally, a dihydropteridine reductase (EC 1.5.1.34).

5. The recombinant microbial cell of any one of the preceding embodiments, wherein at least one nucleic acid sequence encoding a 6-pyruvoyl-tetrahydropterin synthase and at least one nucleic acid sequence encoding a sepiapterin reductase is heterologous.

6. The recombinant microbial cell of any one of the preceding embodiments, wherein at least one nucleic acid sequence encoding a 4a-hydroxytetrahydrobiopterin dehydratase is heterologous.

7. The recombinant microbial cell of any one of the preceding embodiments, wherein each one of said exogenous nucleic acid sequences is operably linked to an inducible, a regulated or a constitutive promoter.

8. The recombinant microbial cell of any one of the preceding embodiments, wherein each one of said exogenous nucleic acid sequences is comprised in a multicopy plasmid or incorporated into a chromosome of the microbial cell.

9. The recombinant microbial cell of any one of the preceding embodiments, which comprises a mutation providing for reduced tryptophan degradation, optionally providing for reduced tryptophanase activity.

10. The recombinant microbial cell of any one of the preceding embodiments, which is derived from a microbial host cell which is a bacterial cell, a yeast host cell, a filamentous fungal cell, or an algeal cell.

11. The recombinant microbial cell of embodiment 10, wherein the microbial host cell is of a genus selected from the group consisting of Acinetobacter, Agrobacterium, Alcaligenes, Anabaena, Aspergillus, Bacillus, Bifidobacterium, Brevibacterium, Candida, Chlorobium, Chromatium, Corynebacteria, Cytophaga, Deinococcus, Enterococcus, Erwinia, Erythrobacter, Escherichia, Flavobacterium, Hansenula, Klebsiella, Lactobacillus, Methanobacterium, Methylobacter, Methylococcus, Methylocystis, Methylomicrobium, Methylomonas, Methylosinus, Mycobacterium, Myxococcus, Pantoea, Phaffia, Pichia, Pseudomonas, Rhodobacter, Rhodococcus, Saccharomyces, Salmonella, Sphingomonas, Streptococcus, Streptomyces, Synechococcus, Synechocystis, Thiobacillus, Trichoderma, Yarrowia, and Zymomonas.

12. The recombinant microbial cell of any preceding embodiment, which is a bacterial cell.

13. The recombinant cell of embodiment 12, which is an Escherichia cell.

14. The recombinant microbial cell of embodiment 13, which is an Escherichia coli cell.

15. The recombinant microbial cell of any one of embodiments 13 and 14, which comprises a mutation in or a deletion of the tnaA gene.

16. The recombinant microbial cell of any one of embodiments 1 to 11, which is a fungal cell.

17. The recombinant microbial cell of embodiment 16, which is a yeast cell.

18. The recombinant microbial cell of embodiment 17, which is a Saccharomyces cell.

19. The recombinant microbial cell of embodiment 18, which is a Saccharomyces cerevisiae cell.

20. The recombinant microbial cell of any preceding embodiment, wherein the L-tryptophan hydroxylase is an L-tryptophan hydroxylase 1 or a catalytically active fragment thereof.

21. The recombinant microbial cell of any preceding embodiment, wherein the L-tryptophan hydroxylase comprises an amino acid sequence having a sequence identity of at least 70%, such as at least 80% or at least 90% to the amino acid sequence of at least one of SEQ ID NOS:1 to 8, or to a catalytically active fragment thereof.

22. The recombinant microbial cell of any preceding embodiment, wherein the L-tryptophan hydroxylase comprises the amino acid sequence of SEQ ID NO:9.

23. The recombinant microbial cell of any one of embodiments 3-22, wherein
(a) the GTP cyclohydrolase I comprises the amino acid sequence of any one of SEQ ID NOS:10-16;
(b) the 6-pyruvoyl-tetrahydropterin synthase comprises the amino acid sequence of any one of SEQ ID NOS:17-22;
(c) the sepiapterin reductase comprises the amino acid sequence of any one of SEQ ID NOS:23-28; or
(d) any combination of (a) to (c).

24. The recombinant microbial cell of any one of embodiments 4 to 23, wherein
(a) the 4a-hydroxytetrahydrobiopterin dehydratase comprises the amino acid sequence of any one of SEQ ID NOS:29-33;
(b) the dihydropteridine reductase comprises the amino acid sequence encoded by SEQ ID NO:34-39; or
(c) a combination of (a) and (b).

25. A microbial cell of any one of the preceding embodiments for use in a method of producing 5-hydroxytryptophan (5HTP), the method comprising culturing the microbial cell in a medium comprising a carbon source.

26. A vector comprising a nucleic acid sequence encoding an L-tryptophan hydroxylase and a nucleic acid sequence encoding one or more enzymes selected from
(a) a GTP cyclohydrolase I (EC 3.5.4.16);
(b) a 6-pyruvoyl-tetrahydropterin synthase (EC 4.2.3.12);
(c) a sepiapterin reductase (EC 1.1.1.153);
(d) a 4a-hydroxytetrahydrobiopterin dehydratase (EC 4.2.1.96);
(e) a dihydropteridine reductase (EC 1.5.1.34);
(f) a combination of any one or more of (a) to (e); or
(g) a combination of at least (b), (c) and (e).

27. The vector of embodiment 26, comprising nucleic acid sequences encoding a GTP cyclohydrolase I, a 6-pyruvoyl-tetrahydropterin synthase and a sepiapterin reductase.

28. The vector of any one of embodiments 26 to 27, comprising nucleic acid sequences encoding a 4a-hydroxytetrahydrobiopterin dehydratase and a dihydropteridine reductase.

29. The vector of embodiment 26, comprising nucleic acid sequences encoding all of (a) to (e).

30. The vector of any one of embodiments 26 to 29, wherein each one of said nucleic acid sequences is operably linked to an inducible, a regulated or a constitutive promoter.

31. The vector of any one of embodiments 26 to 30, which is a plasmid.

32. The vector of any one of embodiments 26 to 31, wherein the nucleic acid sequence encoding an L-tryptophan hydroxylase encodes an amino acid sequence having a sequence identity of at least 70%, such as at least 80% or at least 90%, to the amino acid sequence of any one or more of SEQ ID NOS:1 to 8.

33. The vector of any one of embodiments 26 to 32, wherein the L-tryptophan hydroxylase comprises the amino acid sequence of SEQ ID NO:9.

34. The vector of any one of embodiments 26 to 33, wherein
(a) the GTP cyclohydrolase I comprises the amino acid sequence of any one of SEQ ID NOS:10-16;
(b) the 6-pyruvoyl-tetrahydropterin synthase comprises the amino acid sequence of any one of SEQ ID NOS:17-22;
(c) the sepiapterin reductase comprises the amino acid sequence of any one of SEQ ID NOS:23-28; or
(d) any combination of (a) to (c).

35. The vector of any one of embodiments 26 to 34, wherein
(a) the 4a-hydroxytetrahydrobiopterin dehydratase comprises the amino acid sequence of any one of SEQ ID NOS:29-33;
(b) the dihydropteridine reductase comprises the amino acid sequence encoded by SEQ ID NO:34-39; or
(c) a combination of (a) and (b).

36. A vector comprising nucleic acids encoding an L-tryptophan hydroxylase, a 4a-hydroxytetrahydrobiopterin dehydratase, and a dihydropteridine reductase.

37. The vector of embodiment 36, further comprising nucleic acids encoding a GTP cyclohydrolase I (EC 3.5.4.16), a 6-pyruvoyl-tetrahydropterin synthase, and a sepiapterin reductase.

38. The vector of any one of embodiments 26 to 37, further comprising one or more operably linked regulatory control elements, selection markers, or both.

39. The vector of any one of embodiments 26 to 38, comprising the sequence of SEQ ID NO:61, 92 or 93.

40. A recombinant microbial host cell transformed with the vector of any one of embodiments 26 to 39.

41. The recombinant microbial host cell of embodiment 40, which is derived from a host cell of a genus selected from the group consisting of Acinetobacter, Agrobacterium, Alcaligenes, Anabaena, Aspergillus, Bacillus, Bifidobacterium, Brevibacterium, Candida, Chlorobium, Chromatium, Corynebacteria, Cytophaga, Deinococcus, Enterococcus, Erwinia, Erythrobacter, Escherichia, Flavobacterium, Hansenula, Klebsiella, Lactobacillus, Methanobacterium, Methylobacter, Methylococcus, Methylocystis, Methylomicrobium, Methylomonas, Methylosinus, Mycobacterium, Myxococcus, Pantoea, Phaffia, Pichia, Pseudomonas, Rhodobacter, Rhodococcus, Saccharomyces, Salmonella, Sphingomonas, Streptococcus, Streptomyces, Synechococcus, Synechocystis, Thiobacillus, Trichoderma, Yarrowia, and Zymomonas.

42. A method of producing 5HTP, comprising culturing the recombinant microbial cell of any one of embodiments 1 to 25 and 40 to 41 in a medium comprising a carbon source, and, optionally, isolating 5HTP.

43. The method of embodiment 42, comprising isolating 5HTP and, optionally, purifying 5HTP.

44. A method for preparing a composition comprising 5HTP comprising the steps of:
(a) culturing a microbial cell comprising an exogenous nucleic acid encoding a L-tryptophan hydroxylase and at least one source of THB in a medium comprising a carbon source, optionally in the presence of tryptophan;
(b) isolating 5-hydroxytryptophan;
(c) purifying the isolated 5HTP; and
(d) adding any excipients to obtain a composition comprising 5HTP.

45. The method of embodiment 44, wherein the microbial cell comprises enzymes of a pathway regenerating THB from 4a-hydroxytetrahydrobiopterin.

46. The method of any one of embodiments 44 or 45, wherein the source of THB comprises exogenously added THB.

47. The method of any one of embodiments 44 to 46, wherein the source of THB comprises enzymes of a pathway producing THB from GTP.

48. The method of any one of embodiments 42 to 47, wherein the carbon source is selected from the group consisting of glucose, fructose, sucrose, xylose, mannose, galactose, rhamnose, arabinose, fatty acids, glycerine, starch, glycogen, amylopectin, amylose, cellulose, cellulose acetate, cellulose nitrate, hemicellulose, xylan, glucuronoxylan, arabinoxylan, glucomannan, xyloglucan, lignin, and lignocellulose.

49. A method of producing a recombinant microbial cell, comprising transforming a microbial host cell with one or more vectors comprising nucleic acid sequences encoding
(a) an L-tryptophan hydroxylase (EC 1.14.16.4);
(b) a GTP cyclohydrolase I (EC 3.5.4.16);
(c) a 6-pyruvoyl-tetrahydropterin synthase (EC 4.2.3.12);
(d) a sepiapterin reductase (EC 1.1.1.153);
(e) a 4a-hydroxytetrahydrobiopterin dehydratase (EC 4.2.1.96); and
(f) a dihydropteridine reductase (EC 1.5.1.34),
   each one of said nucleic acid sequences being operably linked to an inducible, a regulated or a constitutive promoter, thereby obtaining the recombinant microbial cell.

50. The method of embodiment 49, wherein the L-tryptophan hydroxylase is a tryptophan hydroxylase 1.

51. The method of any one of embodiments 49 and 50, comprising mutating the cell to reduce tryptophan degradation, optionally to reduce tryptophanase activity.

52. The method of embodiment 51, comprising mutating or deleting a gene encoding a tryptophanase, optionally the tnaA gene.

53. A composition comprising 5HTP obtainable by culturing a recombinant microbial cell comprising an exogenous nucleic acid sequence encoding an L-tryptophan hydroxylase and a source of THB in a medium comprising a carbon source.

54. A method for reducing degradation of 5HTP in a microbial cell comprising tryptophanase activity, comprising mutating the cell to reduce the tryptophanase activity.

55. The method of embodiment 54, comprising mutating or deleting a gene encoding a tryptophanase.

56. The method of any one of embodiments 54 and 55, wherein the microbial host cell is a bacterial cell, a yeast host cell, a filamentous fungal cell, or an algeal cell.

57. The method of embodiment 56, wherein the microbial host cell is of a genus selected from the group consisting of Acinetobacter, Agrobacterium, Alcaligenes, Anabaena, Aspergillus, Bacillus, Bifidobacterium, Brevibacterium, Candida, Chlorobium, Chromatium, Corynebacteria, Cytophaga, Deinococcus, Enterococcus, Erwinia, Erythrobacter, Escherichia, Flavobacterium, Hansenula, Klebsiella, Lactobacillus, Methanobacterium, Methylobacter, Methylococcus, Methylocystis, Methylomicrobium, Methylomonas, Methylosinus, Mycobacterium, Myxococcus, Pantoea, Phaffia, Pichia, Pseudomonas, Rhodobacter, Rhodococcus, Saccharomyces, Salmonella, Sphingomonas, Streptococcus, Streptomyces, Synechococcus, Synechocystis, Thiobacillus, Trichoderma, Yarrowia, and Zymomonas.

58. The method of embodiment 57, wherein the cell is an Escherichia cell.

59. The method of embodiment 58, wherein the cell is an Escherichia coli cell.

60. A microbial cell obtained by the method of any one of embodiments 54 to 59.

1. A recombinant microbial cell comprising an exogenous nucleic acid sequence encoding an L-tryptophan hydroxylase (TPH) (EC 1.14.16.4), and exogenous nucleic acid sequences encoding enzymes of at least one pathway for producing tetrahydrobiopterin (THB).

2. The recombinant microbial cell of claim 1, comprising exogenous nucleic acid sequences encoding enzymes of a first pathway producing THB from guanosin triphosphate (GTP), of a second pathway regenerating THB from 4a-hydroxytetrahydrobiopterin, or of both the first and the second pathway.

3. The recombinant microbial cell of any one of the preceding claims, comprising exogenous nucleic acid sequences encoding
(a) optionally, a GTP cyclohydrolase I (EC 3.5.4.16);
(b) a 6-pyruvoyl-tetrahydropterin synthase (EC 4.2.3.12); and
(c) a sepiapterin reductase (EC 1.1.1.153).

4. The recombinant microbial cell of any one of the preceding claims, comprising exogenous nucleic acid sequences encoding
(a) a 4a-hydroxytetrahydrobiopterin dehydratase (EC 4.2.1.96); and
(b) optionally, a dihydropteridine reductase (EC 1.5.1.34).

5. The recombinant microbial cell of any one of the preceding claims, wherein each one of said exogenous nucleic acid sequences is operably linked to an inducible, a regulated or a constitutive promoter.

6. The recombinant microbial cell of any one of the preceding claims, which comprises a mutation providing for reduced tryptophanase activity.

7. The recombinant microbial cell of any one of the preceding claims, which is derived from a microbial host cell which is a bacterial cell, a yeast host cell, a filamentous fungal cell, or an algeal cell.

8. The recombinant microbial cell of any one of the preceding claims, which is an Escherichia coli cell.

9. The recombinant microbial cell of claim 8, which comprises a mutation in or a deletion of the tnaA gene.

10. The recombinant microbial cell of any one of claims 1 to 7, which is a Saccharomyces cerevisiae cell.

11. The recombinant microbial cell of any one of the preceding claims, wherein the L-tryptophan hydroxylase comprises the amino acid sequence of SEQ ID NO:9.

12. The recombinant microbial cell of any one of claims 3 to 11, wherein
(a) the GTP cyclohydrolase I comprises the amino acid sequence of any one of SEQ ID NOS:10-16;
(b) the 6-pyruvoyl-tetrahydropterin synthase comprises the amino acid sequence of any one of SEQ ID NOS:17-22;
(c) the sepiapterin reductase comprises the amino acid sequence of any one of SEQ ID NOS:23-28; or
(d) any combination of (a) to (c).

13. The recombinant microbial cell of any one of claims 4-12, wherein
(a) the 4a-hydroxytetrahydrobiopterin dehydratase comprises the amino acid sequence of any one of SEQ ID NOS:29-33;
(b) the dihydropteridine reductase comprises the amino acid sequence encoded by SEQ ID NO:34-39; or
(c) a combination of (a) and (b).

14. A vector comprising nucleic acids encoding an L-tryptophan hydroxylase, a 4a-hydroxytetrahydrobiopterin dehydratase, and a dihydropteridine reductase.

15. The vector of claim 14, further comprising nucleic acids encoding a GTP cyclohydrolase I (EC 3.5.4.16), a 6-pyruvoyl-tetrahydropterin synthase, and a sepiapterin reductase.

16. A method of producing 5HTP, comprising culturing the recombinant microbial cell of any one of claims 1 to 13 in a medium comprising a carbon source, and isolating 5HTP.

17. The method of claim 16, wherein the carbon source is selected from the group consisting of glucose, fructose, sucrose, xylose, mannose, galactose, rhamnose, arabinose, fatty acids, glycerine, starch, glycogen, amylopectin, amylose, cellulose, cellulose acetate, cellulose nitrate, hemicellulose, xylan, glucuronoxylan, arabinoxylan, glucomannan, xyloglucan, lignin, and lignocellulose.

18. A method of producing a recombinant microbial cell, comprising transforming a microbial host cell with one or more vectors comprising nucleic acid sequences encoding
(a) an L-tryptophan hydroxylase (EC 1.14.16.4);
(b) a GTP cyclohydrolase I (EC 3.5.4.16);
(c) a 6-pyruvoyl-tetrahydropterin synthase (EC 4.2.3.12);
(d) a sepiapterin reductase (EC 1.1.1.153);
(e) a 4a-hydroxytetrahydrobiopterin dehydratase (EC 4.2.1.96); and
(f) a dihydropteridine reductase (EC 1.5.1.34),
each one of said nucleic acid sequences being operably linked to an inducible, a regulated or a constitutive promoter, thereby obtaining the recombinant microbial cell.


SEQUENCE LISTING (109 sequences)

SEQ ID NO: 1 - protein, 444 aa, Oryctolagus cuniculus
MIEDNKENKDHSLERGRATLIFSLKNEVGGLIKALKIFQEKHVNLLHIESRKSKRRNSEFEIFVDCDTNREQLNDIFHLLKSHTNVLSVTPPDNFTMKEEGMESVPWFPKKISDLDHCANRVLMYGSELDADHPGFKDNVYRKRRKYFADLAMSYKYGDPIPKVEFTEEEIKTWGTVFRELNKLYPTHACREYLKNLPLLSKYCGYREDNIPQLEDISNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHVLSGHAKVKPFDPKITYKQECLITTFQDVYFVSESFEDAKEKMREFTKTIKRPFGVKYNPYTRSIQILKDAKSITNAMNELRHDLDVVSDALGKVSRQLSV

SEQ ID NO: 2 - protein, 444 aa, Homo sapiens
MIEDNKENKDHSLERGRASLIFSLKNEVGGLIKALKIFQEKHVNLLHIESRKSKRRNSEFEIFVDCDINREQLNDIFHLLKSHTNVLSVNLPDNFTLKEDGMETVPWFPKKISDLDHCANRVLMYGSELDADHPGFKDNVYRKRRKYFADLAMNYKHGDPIPKVEFTEEEIKTWGTVFQELNKLYPTHACREYLKNLPLLSKYCGYREDNIPQLEDVSNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHALSGHAKVKPFDPKITCKQECLITTFQDVYFVSESFEDAKEKMREFTKTIKRPFGVKYNPYTRSIQILKDTKSITSAMNELQHDLDVVSDALAKVSRKPSI

SEQ ID NO: 3 - protein, 466 aa, Homo sapiens
MIEDNKENKDHSLERGRASLIFSLKNEVGGLIKALKIFQEKHVNLLHIESRKSKRRNSEFEIFVDCDINREQLNDIFHLLKSHTNVLSVNLPDNFTLKEDGMETVPWFPKKISDLDHCANRVLMYGSELDADHPGFKDNVYRKRRKYFADLAMNYKHGDPIPKVEFTEEEIKTWGTVFQELNKLYPTHACREYLKNLPLLSKYCGYREDNIPQLEDVSNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHALSGHAKVKPFDPKITCKQECLITTFQDVYFVSESFEDAKEKMREFTKTIKRPFGVKYNPYTRSIQILKDTKSITSAMNELQHDLDVVSDALAKSLNEDVLQVSVFALLLFLPSLHGECHPDT

SEQ ID NO: 4 - protein, 502 aa, Bos taurus
MQPAMMMFSSKYWARRGLSLDSAVPEEHQLLTSLTLNKTNSGKNDDKKGNKGSSKNDTATESGKTAVVFSLKNEVGGLVKALKLFQEKHVNMIHIESRKSRRRSSEVEIFVDCECGKTEFNELIQSLKFQTTIVTLNPPENIWTEEEGKLTCVAKGKELEDVPWFPRKISELDRCSHRVLMYGSELDADHPGFKDNVYRQRRKYFVDVAMGYKYGQPIPRVEYTEEETKTWGVVFRELSKLYPTHACREYLKNFPLLTKHCGYREDNVPQLEDVAAFLKERSGFTVRPVAGYLSPRDFLAGLAYRVFHCTQYVRHGSDPLYTPEPDVTLSLLSHVPLIFDDQFPTSFSNEVGRAVILASWGDKQENNQCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRSDLNTVCDALNKMNQYLGI

SEQ ID NO: 5 - protein, 497 aa, Sus scrofa
MQPAMMMFSSKYWARRGLSLDSAVPEEHQLLGSLTVSTFLKLNKSNSGKNDDKKGNKGSGKSDTATESGKTAVVFSLKNEVGGLVKALKLFQEKHVNMVHIESRKSRRRSSEVEIFVDCECGKTEFNELIQSLKFQTTIVTLNPPENIWTEEEELEDVPWFPRKISELDKCSHRVLMYGSELDADHPGFKDNVYRQRRKYFVDLAMGYKYGQPIPRVEYTEEETKTWGIVFRELSKLYPTHACREYLKNFPLLTKYCGYREDNVPQLEDVSVFLKERSGFTVRPVAGYLSPRDFLAGLAYRVFHCTQYVRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRSDLNTVCDALNKMNQYLGI

SEQ ID NO: 6 - protein, 445 aa, Gallus gallus
MIEDNKENKDHAPERGRTAIIFSLKNEVGGLVKALKLFQEKHVNLVHIESRKSKRRNSEFEIFVDCDSNREQLNEIFQLLKSHVSIVSMNPTEHFNVQEDGDMENIPWYPKKISDLDKCANRVLMYGSDLDADHPGFKDNVYRKRRKYFADLAMNYKHGDPIPEIEFTEEEIKTWGTVYRELNKLYPTHACREYLKNLPLLTKYCGYREDNIPQLEDVSRFLKERTGFTIRPVAGYLSPRDFLAGLAFRVFHCTQYVRHSSDPLYTPEPDTCHELLGHVPLLAEPSFAQFSQEIGLASLGASDEAVQKLATCYFFTVEFGLCKQEGQLRVYGAGLLSSISELKHSLSGSAKVKPFDPKVTCKQECLITTFQEVYFVSESFEEAKEKMREFAKTIKRPFGVKYNPYTQSVQILKDTKSIASVVNELRHELDIVSDALSKMGKQLEV

SEQ ID NO: 7 - protein, 447 aa, Mus musculus
MIEDNKENKENKDHSSERGRVTLIFSLENEVGGLIKVLKIFQENHVSLLHIESRKSKQRNSEFEIFVDCDISREQLNDIFPLLKSHATVLSVDSPDQLTAKEDVMETVPWFPKKISDLDFCANRVLLYGSELDADHPGFKDNVYRRRRKYFAELAMNYKHGDPIPKIEFTEEEIKTWGTIFRELNKLYPTHACREYLRNLPLLSKYCGYREDNIPQLEDVSNFLKERTGFSIRPVAGYLSPRDFLSGLAFRVFHCTQYVRHSSDPLYTPEPDTCHELLGHVPLLAEPSFAQFSQEIGLASLGASEETVQKLATCYFFTVEFGLCKQDGQLRVFGAGLLSSISELKHALSGHAKVKPFDPKIACKQECLITSFQDVYFVSESFEDAKEKMREFAKTVKRPFGLKYNPYTQSVQVLRDTKSITSAMNELRYDLDVISDALARVTRWPSV

SEQ ID NO: 8 - protein, 491 aa, Equus caballus
MQPAMMMFSSKYWARRGFSLDSAVPEEHQLLGNLTVNKSNSGKNDDKKGNKGSSRSETAPDSGKTAVVFSLRNEVGGLVKALKLFQEKHVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMSYKYGQPIPRVEYTEEETKTWGVVFRELSRLYPTHACQEYLKNFPLLTKYCGYREDNVPQLEDVSMFLKERSGFAVRPVAGYLSPRDFLAGLAYRVFHCTQYVRHSSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMREFAKSITRPFSVHFNPYTQSVEVLKDSRSIESVVQDLRSDLNTVCDALNKMNQYLGV

SEQ ID NO: 9 - protein, 315 aa, Oryctolagus cuniculus
MESVPWFPKKISDLDHCANRVLMYGSELDADHPGFKDNVYRKRRKYFADLAMSYKYGDPIPKVEFTEEEIKTWGTVFRELNKLYPTHACREYLKNLPLLSKYCGYREDNIPQLEDISNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHVLSGHAKVKPFDPKITYKQECLITTFQDVYFVSESFEDAKEKMREFTKTIKRPFGVKYNPYTRSIQILKDAKS

SEQ ID NO: 10 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 11 - protein, 241 aa, Mus musculus
MEKPRGVRCTNGFSERELPRPGASPPAEKSRPPEAKGAQPADAWKAGRHRSEEENQVNLPKLAAAYSSILLSLGEDPQRQGLLKTPWRAATAMQYFTKGYQETISDVLNDAIFDEDHDEMVIVKDIDMFSMCEHHLVPFVGRVHIGYLPNKQVLGLSKLARIVEIYSRRLQVQERLTKQIAVAITEALQPAGVGVVIEATHMCMVMRGVQKMNSKTVTSTMLGVFREDPKTREEFLTLIRS

SEQ ID NO: 12 - protein, 222 aa, Escherichia coli
MPSLSKEAALVHEALVARGLETPLRPPVHEMDNETRKSLIAGHMTEIMQLLNLDLADDSLMETPHRIAKMYVDEIFSGLDYANFPKITLIENKMKVDEMVTVRDITLTSTCEHHFVTIDGKATVAYIPKDSVIGLSKINRIVQFFAQRPQVQERLTQQILIALQTLLGTNNVAVSIDAVHYCVKARGIRDATSATTTTSLGGLFKSSQNTRHEFLRAVRHHN

SEQ ID NO: 13 - protein, 243 aa, Saccharomyces cerevisiae
MHNIQLVQEIERHETPLNIRPTSPYTLNPPVERDGFSWPSVGTRQRAEETEEEEKERIQRISGAIKTILTELGEDVNREGLLDTPQRYAKAMLYFTKGYQTNIMDDVIKNAVFEEDHDEMVIVRDIEIYSLCEHHLVPFFGKVHIGYIPNKKVIGLSKLARLAEMYARRLQVQERLTKQIAMALSDILKPLGVAVVMEASHMCMVSRGIQKTGSSTVTSCMLGGFRAHKTREEFLTLLGRRSI

SEQ ID NO: 14 - protein, 190 aa, Bacillus subtilis
MKEVNKEQIEQAVRQILEAIGEDPNREGLLDTPKRVAKMYAEVFSGLNEDPKEHFQTIFGENHEELVLVKDIAFHSMCEHHLVPFYGKAHVAYIPRGGKVTGLSKLARAVEAVAKRPQLQERITSTIAESIVETLDPHGVMVVVEAEHMCMTMRGVRKPGAKTVTSAVRGVFKDDAAARAEVLEHIKRQD

SEQ ID NO: 15 - protein, 201 aa, Streptomyces avermitilis
MTDPVTLDGEGTIGEFDEKRAENAVRELLIAVGEDPDREGLRETPGRVARAYREIFAGLWQKPEDVLTTTFDIGHDEMVLVKDIEVLSSCEHHLVPFVGVAHVGYIPSTDGKITGLSKLARLVDVYARRPQVQERLTTQVADSLMEILEPRGVIVVVECEHMCMSMRGVRKPGAKTITSAVRGQLRDPATRNEAMSLIMAR

SEQ ID NO: 16 - protein, 222 aa, Salmonella typhi
MPSLSKEAALVHDALVARGLETPLRPPMDELDNETRKSLIAGHMTEIMQLLNLDLSDDSLMETPHRIAKMYVDEIFAGLDYANFPKITLIENKMKVDEMVTVRDITLTSTCEHHFVTIDGKATVAYIPKDSVIGLSKINRIVQFFAQRPQVQERLTQQILTALQTLLGTNNVAVSIDAVHYCVKARGIRDATSATTTTSLGGLFKSSQNTRQEFLRAVRHHP

SEQ ID NO: 17 - protein, 250 aa, Homo sapiens; identical to SEQ ID NO: 10

SEQ ID NO: 18 - protein, 144 aa, Rattus norvegicus
MNAAVGLRRRARLSRLVSFSASHRLHSPSLSAEENLKVFGKCNNPNGHGHNYKVVVTIHGEIDPVTGMVMNLTDLKEYMEEAIMKPLDHKNLDLDVPYFADVVSTTENVAVYIWENLQRLLPVGALYKVKVYETDNNIVVYKGE

SEQ ID NO: 19 - protein, 124 aa, Bacteroides thetaiotaomicron
MFTVIKRMEISASHKLVLPYRSKCASLHGHNWIITVYCRSSRLNSEGMVVDFTRIKEVVTEKLDHQNLNEVLPFNPTAENIARWVCRQIPQCYKVEVQESEGNIVIYEKDAVANEKTPAAGETE

SEQ ID NO: 20 - protein, 290 aa, Thermosynechococcus elongatus
MNCIIHRRAEFAASHRYWLPEWSEAENLARFGANSRFPGHGHNYELFVSMEGVVDDFGMVLNLSDVKHIIRREVIEPLNFSYLNEVWPEFQATLPTTEHIARVIWDRLFPHLPLVRIRLFEHPRLWADYTGDPMEAYLSVGAHFSAAHRLALEDLSYEENCRIYGKCARPHGHGHNYHVEITVKGSIHPRTGMVVDLVKLEEVLKEQVIEPLDHTFLNKDIPYFATVVPTAENIAIYIAHLLQEPVRQLGATLHRVKLIESPNNSCEILCEELPPRNEVISGALPVLERV

SEQ ID NO: 21 - protein, 147 aa, Streptococcus thermophilus
MFFAPKEIKTETGESLVYNLHRTMVSKEFTFDAAHHLFNYEGKCKSLHGHTYHLQIAVSGYLDDRGMTYDFGDLKNIYKNHLEPYLDHRYLNESLPYMNTTAENMVFWIFQTTSKYLSEERELRLEYVRLYETPTAFAEFRREWLDD

SEQ ID NO: 22 - protein, 291 aa, Acaryochloris marina
MKCLIHRRAEFSASHRYWLPELSKSENQEKFGQCTRSPGHGHNYELFVSMWGELDQYGMVLNLSNVKQVIKREVTAPLNFSYLNEVWPEFKETLPTTEHLARVIWQRLEPHLPIVNIQLFEHPKLWADYKGAGMEAYLTVGSHFSAAHRLALPELSFEENCEIYGKCARPHGHGHNYHLEVTVKGEVDARTGMIVDLVALQSLVDDVVLDPLDHTFLNKDIPYFEKVVPTAENIAFYIAKLLREPILKIGAELHRIKLIESPNNSCEVLCSDLFDTAPMLSGRMGEPALVG

SEQ ID NO: 23 - protein, 261 aa, Homo sapiens
MEGGLGRAVCLLTGASRGFGRTLAPLLASLLSPGSVLVLSARNDEALRQLEAELGAERSGLRVVRVPADLGAEAGLQQLLGALRELPRPKGLQRLLLINNAGSLGDVSKGFVDLSDSTQVNNYWALNLTSMLCLTSSVLKAFPDSPGLNRTVVNISSLCALQPFKGWALYCAGKAARDMLFQVLALEEPNVRVLNYAPGPLDTDMQQLARETSVDPDMRKGLQELKAKGKLVDCKVSAQKLLSLLEKDEFKSGAHVDFYDK

SEQ ID NO: 24 - protein, 262 aa, Rattus norvegicus
MEGGRLGCAVCVLTGASRGFGRALAPQLAGLLSPGSVLLLSARSDSMLRQLKEELCTQQPGLQVVLAAADLGTESGVQQLLSAVRELPRPERLQRLLLINNAGTLGDVSKGFLNINDLAEVNNYWALNLTSMLCLTTGTLNAFSNSPGLSKTVVNISSLCALQPFKGWGLYCAGKAARDMLYQVLAVEEPSVRVLSYAPGPLDTNMQQLARETSMDPELRSRLQKLNSEGELVDCGTSAQKLLSLLQRDTFQSGAHVDFYDI

SEQ ID NO: 25 - protein, 261 aa, Mus musculus
MEADGLGCAVCVLTGASRGFGRALAPQLARLLSPGSVMLVSARSESMLRQLKEELGAQQPDLKVVLAAADLGTEAGVQRLLSAVRELPRPEGLQRLLLINNAATLGDVSKGFLNVNDLAEVNNYWALNLTSMLCLTSGTLNAFQDSPGLSKTVVNISSLCALQPYKGWGLYCAGKAARDMLYQVLAAEEPSVRVLSYAPGPLDNDMQQLARETSKDPELRSKLQKLKSDGALVDCGTSAQKLLGLLQKDTFQSGAHVDFYD

SEQ ID NO: 26 - protein, 267 aa, Bos taurus
MEGSVGKVGGLGRTLCVLTGASRGFGRTLAQVLAPLMSPRSVLVLSARNDEALRQLETELGAEWPGLRIVRVPADLGAETGLQQLVGALCDLPRPEGLQRVLLINNAGTLGDVSKRWVDLTDPTEVNNYWTLNLTSTLCLTSSILQAFPDSPGLSRTVVNISSICALQPFKGWGLYCAGKAARNMMFQVLAAEEPSVRVLSYGPGPLDTDMQQLARETSVDPDLRKSLQELKRKGELVDCKISAQKLLSLLQNDKFESGAHIDFYDE

SEQ ID NO: 27 - protein, 261 aa, Danio rerio
MSTASGFGKALVIITGASRGFGRALALSVAARVSPGSVLVLAARSEEQLLELKSALTRGETGLTVRCVPVDLGCEAGVEKLIAETRDIQPDIQHLLLFHNAASLGDVSRYCRDFTNMEELNSYLSLNVSSALCLTAGVLRTYPKRSGLTRVIVNISSLCALRPFPTWVQYCSGKAARDMMFRVLAEEEPELRVLNYAPGPLDTDMQREARSSCADSKLRNTFSQMHANGQLLTCDQSIQKLMSVLLEDKYSSGEHLDYYDL

SEQ ID NO: 28 - protein, 263 aa, Xenopus laevis
MTAARAGALGSVLCVLTGASRGFGRTLAHELCPRVLPGSTLLLVSRTEEALKGLAEELGHEFPGVRVRWAAADLSTTEGVSATVRAARELQAGTAHRLLIINNAGSIGDVSKMFVDFSAPEEVTEYMKFNVSSPLCLTASLLKTFPRRPDLQRLVVNVSSLAALQPYKSWVLYCSGKAARDMMFRVLAEEEDDVRVLSYAPGPLDTDMHEVACTQTADPELRRAIMDRKEKGNMVDIRVSANKMLDLLEADAYKSGDHIDFYD

SEQ ID NO: 29 - protein, 262 aa, Pseudomonas aeruginosa
MKTTQYVARQPDDNGFIHYPETEHQVWNTLITRQLKVIEGRACQEYLDGIEQLGLPHERIPQLDEINRVLQATTGWRVARVPALIPFQTFFELLASQQFPVATFIRTPEELDYLQEPDIFHEIFGHCPLLTNPWFAEFTHTYGKLGLKASKEERVFLARLYWMTIEFGLVETDQGKRIYGGGILSSPKETVYSLSDEPLHQAFNPLEAMRTPYRIDILQPLYFVLPDLKRLFQLAQEDIMALVHEAMRLGLHAPLFPPKQAA

SEQ ID NO: 30 - protein, 104 aa, Bacillus cereus var. anthracis
MMLRLTEEEVQEELLKLDKWVVKDEKWIERKYMFSDYLKGVEFVSEAAKLSEEHNHHPFILIQYKAVIITLSSWNAKGLTKLDFELAKQFDELFVQNEKAVIRK

SEQ ID NO: 31 - protein, 188 aa, Corynebacterium genitalium
MSDTLDALDIHEPDEAFLMATEAEVEVPSQPCALAVLVSDHKQGGAIDEGTDRLVFELLQEIGFKVDGVVYVKSKKSEIRKVIETAVVGGVDLVVTVGGTGVGPRDKAPEATRGVIDQLVPGVAQAVRASGQACGAVDACTSRGICGVSGSTVVVNLAPSRAAIRDGISTISPLVAHLISELRKYSVQ

SEQ ID NO: 32 - protein, 63 aa, Lactobacillus ruminis
MVKLFPSENARRWHRWNHEVLLLVNIQCSLKQPLWSAEGKVDKNREKCAAFVYRLVEIQDARI

SEQ ID NO: 33 - protein, 96 aa, Rhodobacteraceae bacterium
MSERLFDDTRGPLLDPLFATGWAMVEGRDAIEKHYKFKNFADAFGWMTRAAIWSEKWDHHPEWLNVYNKVHVVLTTHSVDGLSPLDVKLARKFDSL

SEQ ID NO: 34 - protein, 244 aa, Homo sapiens
MAAAAAAGEARRVLVYGGRGALGSRCVQAFRARNWWVASVDVVENEEASASIIVKMTDSFTEQADQVTAEVGKLLGEEKVDAILCVAGGWAGGNAKSKSLFKNCDLMWKQSIWTSTISSHLATKHLKEGGLLTLAGAKAALDGTPGMIGYGMAKGAVHQLCQSLAGKNSGMPPGAAAIAVLPVTLDTPMNRKSMPEADFSSWTPLEFLVETFHDWITGKNRPSSGSLIQVVTTEGRTELTPAYF

SEQ ID NO: 35 - protein, 241 aa, Rattus norvegicus
MAASGEARRVLVYGGRGALGSRCVQAFRARNWWVASIDVVENEEASASVIVKMTDSFTEQADQVTAEVGKLLGDQKVDAILCVAGGWAGGNAKSKSLFKNCDLMWKQSIWTSTISSHLATKHLKEGGLLTLAGAKAALDGTPGMIGYGMAKGAVHQLCQSLAGKNSGMPSGAAAIAVLPVTLDTPMNRKSMPEADFSSWTPLEFLVETFHDWITGNKRPNSGSLIQVVTTDGKTELTPAYF

SEQ ID NO: 36 - protein, 243 aa, Sus scrofa
MAAAAAGEARRVLVYGGRGALGSRCVQAFRARNWWVASIDVVENEEASANVVVKMTDSFTEQADQVTAEVGKLLGTEKVDAILCVAGGWAGGNAKSKSLFKNCDLMWKQSMWTSTISSHLATKHLKEGGLLTLAGAKAALDGTPGMIGYGMAKGAVHQLCQSLAGKDSGMPSGAAAIAVLPVTLDTPLNRKSMPHADFSSWTPLEFLVETFHDWIIEKNRPSSGSLIQVVTTQGKTELTPAYF

SEQ ID NO: 37 - protein, 242 aa, Bos taurus
MAAAAGEARRVLVYGGRGALGSRCVQAFRARNWWVASIDVQENEEASANVVVKMTDSFTEQADQVTAEVGKLLGTEKVDAILCVAGGWAGGNAKSKSLFKNCDLMWKQSVWTSTISSHLATKHLKEGGLLTLAGARAALDGTPGMIGYGMAKAAVHQLCQSLAGKSSGLPPGAAAVALLPVTLDTPVNRKSMPEADFSSWTPLEFLVETFHDWITEKNRPSSGSLIQVVTTEGKTELTAASP

SEQ ID NO: 38 - protein, 396 aa, Escherichia coli
MLDAQTIATVKATIPLLVETGPKLTAHFYDRMFTHNPELKEIFNMSNQRNGDQREALFNAIAAYASNIENLPALLPAVEKIAQKHTSFQIKPEQYNIVGEHLLATLDEMFSPGQEVLDAWGKAYGVLANVFINREAEIYNENASKAGGWEGTRDFRIVAKTPRSALITSFELEPVDGGAVAEYRPGQYLGVWLKPEGFPHQEIRQYSLTRKPDGKGYRIAVKREEGGQVSNWLHNHANVGDVVKLVAPAGDFFMAVADDTPVTLISAGVGQTPMLAMLDTLAKAGHTAQVNWFHAAENGDVHAFADEVKELGQSLPRFTAHTWYRQPSEADRAKGQFDSEGLMDLSKLEGAFSDPTMQFYLCGPVGFMQFAAKQLVDLGVKQENIHYECFGPHKVL

SEQ ID NO: 39 - protein, 231 aa, Dictyostelium discoideum
MSKNILVLGGSGALGAEVVKFFKSKSWNTISIDFRENPNADHSFTIKDSGEEEIKSVIEKINSKSIKVDTFVCAAGGWSGGNASSDEFLKSVKGMIDMNLYSAFASAHIGAKLLNQGGLFVLTGASAALNRTSGMIAYGATKAATHHIIKDLASENGGLPAGSTSLGILPVTLDTPTNRKYMSDANFDDWTPLSEVAEKLFEWSTNSDSRPTNGSLVKFETKSKVTTWTNL

SEQ ID NO: 40 - DNA, 948 bp, Oryctolagus cuniculus
atggagagtgttccttggtttccaaagaagatttcagacctggaccattgtgctaaccgagttctgatgtatggatctgagctagatgcagaccaccctggcttcaaagacaatgtctaccgtaaaagacgaaagtactttgcagactcggctatgagctataaatatggagaccccattcctaaggttgaattcacggaagaggagattaagacctggggaaccgtattccgggagctcaacaaactctatccgacccatgcttgcagagagtatctcaaaaatttacctctgctttccaagtattgtggatatcaggaagacaatatcccacagctggaagatatttcaaactttttaaaagagcgcacaggtttttccattcgtcctgtggctggttacttatcaccaagagatttcttatcaggtttagcctttcgagtttttcactgcactcaatatgtgagacacagttcagaccccttctataccccagagccggatacctgccatgaactcttaggtcacgttccccttttggctgagccaagttttgctcagttctcccaagaaattggcctggcttcccttggagcttcagaggaggctgttcaaaaactggcaacgtgctactttttcactgtggagtttggtctatgtaaacaagacggacagttacgagtcttcggcgctggcttactttcttctatcagtgaactcaaacatgtgctttctggacatgccaaagtaaagccttttgatcccaagattacgtacaaacaagaatgcctcatcacaacttttcaggatgtctactttgtatctgaaagctttgaagatgcaaaggagaagatgagagaatttaccaaaacaattaagcgtccctttggagtgaaatataatccctacacacgaagcattcagatcctgaaagacgccaaaagctaa

SEQ ID NO: 41 - DNA, 669 bp, Escherichia coli
atgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtgatcggtctgtcaaaaattaaccgcattgtgcagttctttgcccagcgtccgcaggtgcaggaacgtctgacgcagcaaattcttattgcgctacaaacgctgctgggcaccaataacgtggctgtctcgatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactaa

SEQ ID NO: 42 - DNA, 435 bp, Rattus norvegicus
atgaacgcggcggttggccttcggcgccgcgcgcgattgtcgcgcctcgtgtccttcagcgcgagccaccggctgcacagcccatctctgagtgctgaggagaacttgaaagtgtttgggaaatgcaacaatccgaatggccatgggcacaactataaagttgtggtgacaattcatggagagatcgatccggttacaggaatggttatgaatttgactgacctcaaagaatacatggaggaggccattatgaagccccttgatcacaagaacctggatctggatgtgccatactttgcagatgttgtaagcacgacagaaaatgtagctgtctatatctgggagaacctgcagagacttcttccagtgggagctctctataaagtaaaagtgtatgaaactgacaacaacattgtggtctacaaaggagaataa

SEQ ID NO: 43 - DNA, 789 bp, Rattus norvegicus
atggaaggaggcaggctaggttgcgctgtctgcgtgctgaccggggcttcccggggcttcggccgcgccctggccccgcagctggccgggttgctgtcgcccggttcggtgttgcttctaagcgcacgcagtgactcgatgctgcggcaactgaaggaggagctctgtacgcagcagccgggcctgcaagtggtgctggcagccgccgatttgggcaccgagtccggcgtgcaacagttgctgagcgcggtgcgcgagctccctaggcccgagaggctgcagcgcctcctgctcatcaacaatgcaggcactcttggggatgtttccaaaggcttcctgaacatcaatgacctagctgaggtgaacaactactgggccctgaacctaacctccatgctctgcttgaccaccggcaccttgaatgccttctccaatagccctggcctgagcaagactgtagttaacatctcatctctgtgtgccctgcagcccttcaagggctggggactctactgtgcagggaaggctgcccgagacatgttataccaggtcctggctgttgaggaacccagtgtgagggtgctgagctatgccccaggtcccctggacaccaacatgcagcagttggcccgggaaacctccatggacccagagttgaggagcagactgcagaagttgaattctgagggggagctggtggactgtgggacttcagcccagaaactgctgagcttgctgcaaagggacaccttccaatctggagcccacgtggacttctatgacatttaa

SEQ ID NO: 44 - DNA, 789 bp, Pseudomonas aeruginosa
atgaaaacgacgcagtacgtggcccgccagcccgacgacaacggtttcatccactatccggaaaccgagcaccaggtctggaataccctgatcacccggcaactgaaggtgatcgaaggccgcgcctgtcaggaatacctcgacggcatcgaacagctcggcctgccccacgagcggatcccccagctcgacgagatcaacagggttctccaggccaccaccggctggcgcgtggcgcgggttccggcgctgattccgttccagaccttcttcgaactgctggccagccagcaattccccgtcgccacctttatccgcaccccggaagaactggactacctgcaggagccggacatcttccacgagatcttcggccactgcccactgctgaccaacccctggttcgccgagttcacccatacctacggcaagctcggcctcaaggcgagcaaggaggaacgcgtgttcctcgcccgcctgtactggatgaccatcgagttcggcctggtcgagaccgaccagggcaagcgcatctacggcggcggcatcctctcctcgccgaaggagaccgtctactgcctctccgacgagccgctgcaccaggccttcaatccgctggaggcgatgcgcacgccctaccgcatcgacatcctgcaaccgctctatttcgtcctgcccgacctcaagcgcctgttccaactggcccaggaagacatcatggcactggtccacgaggccatgcgcctgggcctgcacgcgccgctgttcccgcccaagcaggcggcctaa

SEQ ID NO: 45 - DNA, 654 bp, Escherichia coli
atggatatcatttctgtcgccttaaagcgtcattccactaaggcatttgatgccagcaaaaaacttaccccggaacaggccgagcagatcaaaacgctactgcaatacagcccatccagcaccaactcccagccgtggcattttattgttgccagcacggaagaaggtaaagcgcgtgttgccaaatccgctgccggtaattacgtgttcaacgagcgtaaaatgcttgatgcctcgcacgtcgtggtgttctgtgcaaaaaccgcgatggacgatgtctggctgaagctggttgttgaccaggaagatgccgatggccgctttgccacgccggaagcgaaagccgcgaacgataaaggtcgcaagttcttcgctgatatgcaccgtaaagatctgcatgatgatgcagagtggatggcaaaacaggtttatctcaacgtcggtaacttcctgctcggcgtggcggctctgggtctggacgcggtacccatcgaaggttttgacgccgccatcctcgatgcagaatttggtctgaaagagaaaggctacaccagtctggtggttgttccggtaggtcatcacagcgttgaagattttaacgctacgctgccgaaatctcgtctgccgcaaaacatcaccttaaccgaagtgtaa

SEQ ID NO: 46 - DNA, 106 bp, Artificial sequence
  T7 promoter
atctcgatcccgcgaaattaatacgactcactataggggaattgtgagcggataacaattcccctctagaaataattttgtttaactttaagaaggagatatacat

SEQ ID NO: 47 - DNA, 133 bp, Artificial sequence
  T7 terminator sequence
tgagtttgatccggctgctaacaaagcccgaaaggaagctgagttggctgctgccaccgctgagcaataactagcataaccccttggggcctctaaacgggtcttgaggggttttttgctgaaaggaggaact

SEQ ID NO: 48 - DNA, 18 bp, Artificial sequence
  Intragenic region containing an optimized ribosomal binding site
gccgcggaggattacact

SEQ ID NO: 49 - DNA, 216 bp, Artificial sequence
  Linker region 1
gtttccgttcggccggccttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatggcgcgcc

SEQ ID NO: 50 - DNA, 171 bp, Artificial sequence
  Linker region 2
ctggtcattgccaggcaggataaaacgtcgatcaacgctggcatgctctacttttttatcgcccacgccggatcggtgctgataatgatcgccttcttgctgatggggcgcgaaagcggcagcctcgattttgccagtttccgcacgctttcactttctccggggctggcg

SEQ ID NO: 51 - DNA, 5296 bp, Artificial sequence
  Plasmid pTHB
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatccgtttccgttcgcggccgcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatggcgcgccatctcgatcccgcgaaattaatacgactcactataggggaattgtgagcggataacaattcccctctagaaataattttgtttaactttaagaaggagatatacatatgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtgatcggtctgtcaaaaattaaccgcattgtgcagttctttgcccagcgtccgcaggtgcaggaacgtctgacgcagcaaattcttattgcgctacaaacgctgctgggcaccaataacgtggctgtctcgatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactaataagccgcggaggattacactatgaacgcggcggttggccttcggcgccgcgcgcgattgtcgcgcctcgtgtccttcagcgcgagccaccggctgcacagcccatctctgagtgctgaggagaacttgaaagtgtttgggaaatgcaacaatccgaatggccatgggcacaactataaagttgtggtgacaattcatggagagatcgatccggttacaggaatggttatgaatttgactgacctcaaagaatacatggaggaggccattatgaagccccttgatcacaagaacctggatctggatgtgccatactttgcagatgttgtaagcacgacagaaaatgtagctgtctatatctgggagaacctgcagagacttcttccagtgggagctctctataaagtaaaagtgtatgaaactgacaacaacattgtggtctacaaaggagaataataagccgcggaggattacactatggaaggaggcaggctaggttgcgctgtctgcgtgctgaccggggcttcccggggcttcggccgcgccctggccccgcagctggccgggttgctgtcgcccggttcggtgttgcttctaagcgcacgcagtgactcgatgctgcggcaactgaaggaggagctctgtacgcagcagccgggcctgcaagtggtgctggcagccgccgatttgggcaccgagtccggcgtgcaacagttgctgagcgcggtgcgcgagctccctaggcccgagaggctgcagcgcctcctgctcatcaacaatgcaggcactcttggggatgtttccaaaggcttcctgaacatcaatgacctagctgaggtgaacaactactgggccctgaacctaacctccatgctctgcttgaccaccggcaccttgaatgccttctccaatagccctggcctgagcaagactgtagttaacatctcatctctgtgtgccctgcagcccttcaagggctggggactctactgtgcagggaaggctgcccgagacatgttataccaggtcctggctgttgaggaacccagtgtgagggtgctgagctatgccccaggtcccctggacaccaacatgcagcagttggcccgggaaacctccatggacccagagttgaggagcagactgcagaagttgaattctgagggggagctggtggactgtgggacttcagcccagaaactgctgagcttgctgcaaagggacaccttccaatctggagcccacgtggacttctatgacatttaataatgagtttgatccggctgctaacaaagcccgaaaggaagctgagttggctgctgccaccgctgagcaataactagcataaccccttggggcctctaaacgggtcttgaggggttttttgctgaaaggaggaactttcctggtttctggtcattgccaggcaggataaaacgtcgatcaacgctggcatgctctacttttttatcgcccacgccggatcggtgctgataatgatcgccttcttgctgatggggcgcgaaagcggcagcctcgattttgccagtttccgcacgctttcactttctccggggctggcggcggccgcgttcctgctgggtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 52 - DNA, 5768 bp, Artificial sequence
  Plasmid pTRP
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttcctggtttgcggccgctggtcattgccaggcaggataaaacgtcgatcaacgctggcatgctctacttttttatcgcccacgccggatcggtgctgataatgatcgccttcttgctgatggggcgcgaaagcggcagcctcgattttgccagtttccgcacgctttcactttctccggggctggcgtcggcggtgttcctgctggatctcgatcccgcgaaattaatacgactcactataggggaattgtgagcggataacaattcccctctagaaataattttgtttaactttaagaaggagatatacatatggagagtgttccttggtttccaaagaagatttcagacctggaccattgtgctaaccgagttctgatgtatggatctgagctagatgcagaccaccctggcttcaaagacaatgtctaccgtaaaagacgaaagtactttgcagactcggctatgagctataaatatggagaccccattcctaaggttgaattcacggaagaggagattaagacctggggaaccgtattccgggagctcaacaaactctatccgacccatgcttgcagagagtatctcaaaaatttacctctgctttccaagtattgtggatatcaggaagacaatatcccacagctggaagatatttcaaactttttaaaagagcgcacaggtttttccattcgtcctgtggctggttacttatcaccaagagatttcttatcaggtttagcctttcgagtttttcactgcactcaatatgtgagacacagttcagaccccttctataccccagagccggatacctgccatgaactcttaggtcacgttccccttttggctgagccaagttttgctcagttctcccaagaaattggcctggcttcccttggagcttcagaggaggctgttcaaaaactggcaacgtgctactttttcactgtggagtttggtctatgtaaacaagacggacagttacgagtcttcggcgctggcttactttcttctatcagtgaactcaaacatgtgctttctggacatgccaaagtaaagccttttgatcccaagattacgtacaaacaagaatgcctcatcacaacttttcaggatgtctactttgtatctgaaagctttgaagatgcaaaggagaagatgagagaatttaccaaaacaattaagcgtccctttggagtgaaatataatccctacacacgaagcattcagatcctgaaagacgccaaaagctaataagccgcggaggattacactatggatatcatttctgtcgccttaaagcgtcattccactaaggcatttgatgccagcaaaaaacttaccccggaacaggccgagcagatcaaaacgctactgcaatacagcccatccagcaccaactcccagccgtggcattttattgttgccagcacggaagaaggtaaagcgcgtgttgccaaatccgctgccggtaattacgtgttcaacgagcgtaaaatgcttgatgcctcgcacgtcgtggtgttctgtgcaaaaaccgcgatggacgatgtctggctgaagctggttgttgaccaggaagatgccgatggccgctttgccacgccggaagcgaaagccgcgaacgataaaggtcgcaagttcttcgctgatatgcaccgtaaagatctgcatgatgatgcagagtggatggcaaaacaggtttatctcaacgtcggtaacttcctgctcggcgtggcggctctgggtctggacgcggtacccatcgaaggttttgacgccgccatcctcgatgcagaatttggtctgaaagagaaaggctacaccagtctggtggttgttccggtaggtcatcacagcgttgaagattttaacgctacgctgccgaaatctcgtctgccgcaaaacatcaccttaaccgaagtgtaataagccgcggaggattacactatgaaaacgacgcagtacgtggcccgccagcccgacgacaacggtttcatccactatccggaaaccgagcaccaggtctggaataccctgatcacccggcaactgaaggtgatcgaaggccgcgcctgtcaggaatacctcgacggcatcgaacagctcggcctgccccacgagcggatcccccagctcgacgagatcaacagggttctccaggccaccaccggctggcgcgtggcgcgggttccggcgctgattccgttccagaccttcttcgaactgctggccagccagcaattccccgtcgccacctttatccgcaccccggaagaactggactacctgcaggagccggacatcttccacgagatcttcggccactgcccactgctgaccaacccctggttcgccgagttcacccatacctacggcaagctcggcctcaaggcgagcaaggaggaacgcgtgttcctcgcccgcctgtactggatgaccatcgagttcggcctggtcgagaccgaccagggcaagcgcatctacggcggcggcatcctctcctcgccgaaggagaccgtctactgcctctccgacgagccgctgcaccaggccttcaatccgctggaggcgatgcgcacgccctaccgcatcgacatcctgcaaccgctctatttcgtcctgcccgacctcaagcgcctgttccaactggcccaggaagacatcatggcactggtccacgaggccatgcgcctgggcctgcacgcgccgctgttcccgcccaagcaggcggcctaataatgagtttgatccggctgctaacaaagcccgaaaggaagctgagttggctgctgccaccgctgagcaataactagcataaccccttggggcctctaaacgggtcttgaggggttttttgctgaaaggaggaactccatgcgctgttcaaagggctgctatttctcggcgcgggagcgattatttcgcgtttgcatacccacgacatggaaaaaatgggggcactagcgaaacggatgccgtggacagccgcagcatgcctgattggttgcctcgcgatatcagccattcctccgctgaatggttttatcagcgaatggtagcggccgctgcagtcgcgatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 53 - DNA, 80 bp, Artificial sequence
  Primer sequence
ggttgcctcgcgatatcagccattcctccgctgaatggttttatcagcgaatggtaccgggccgtcgaccaattctcatg

SEQ ID NO: 54 - DNA, 80 bp, Artificial sequence
  Primer sequence
atcgaatataacttcgtataatgtatgctatacgaagttattagcgatgagctcggacttccattgttcattccacggac

SEQ ID NO: 55 - DNA, 20 bp, Artificial sequence
  Primer sequence
tcactttacgggtcctttcc

SEQ ID NO: 56 - DNA, 20 bp, Artificial sequence
  Primer sequence
ggccgcttctttactgagtg

SEQ ID NO: 57 - DNA, 20 bp, Artificial sequence
  Primer sequence
ccgctgagcaataactagca

SEQ ID NO: 58 - DNA, 20 bp, Artificial sequence
  Primer sequence
gtattaatttcgcgggatcg

SEQ ID NO: 59 - DNA, 20 bp, Artificial sequence
  Primer sequence
ccgctgagcaataactagca

SEQ ID NO: 60 - DNA, 20 bp, Artificial sequence
  Primer sequence
ggcagttattggtgccctta

SEQ ID NO: 61 - DNA, 12737 bp, Artificial sequence
  Bacterial artificial chromosome
ctcgatcccgcgaaattaatacgactcactataggggaattgtgagcggataacaattcccctctagaaataattttgtttaactttaagaaggagatatacatatgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtgatcggtctgtcaaaaattaaccgcattgtgcagttctttgcccagcgtccgcaggtgcaggaacgtctgacgcagcaaattcttattgcgctacaaacgctgctgggcaccaataacgtggctgtctcgatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactaataagccgcggaggattacactatgaacgcggcggttggccttcggcgccgcgcgcgattgtcgcgcctcgtgtccttcagcgcgagccaccggctgcacagcccatctctgagtgctgaggagaacttgaaagtgtttgggaaatgcaacaatccgaatggccatgggcacaactataaagttgtggtgacaattcatggagagatcgatccggttacaggaatggttatgaatttgactgacctcaaagaatacatggaggaggccattatgaagccccttgatcacaagaacctggatctggatgtgccatactttgcagatgttgtaagcacgacagaaaatgtagctgtctatatctgggagaacctgcagagacttcttccagtgggagctctctataaagtaaaagtgtatgaaactgacaacaacattgtggtctacaaaggagaataataagccgcggaggattacactatggaaggaggcaggctaggttgcgctgtctgcgtgctgaccggggcttcccggggcttcggccgcgccctggccccgcagctggccgggttgctgtcgcccggttcggtgttgcttctaagcgcacgcagtgactcgatgctgcggcaactgaaggaggagctctgtacgcagcagccgggcctgcaagtggtgctggcagccgccgatttgggcaccgagtccggcgtgcaacagttgctgagcgcggtgcgcgagctccctaggcccgagaggctgcagcgcctcctgctcatcaacaatgcaggcactcttggggatgtttccaaaggcttcctgaacatcaatgacctagctgaggtgaacaactactgggccctgaacctaacctccatgctctgcttgaccaccggcaccttgaatgccttctccaatagccctggcctgagcaagactgtagttaacatctcatctctgtgtgccctgcagcccttcaagggctggggactctactgtgcagggaaggctgcccgagacatgttataccaggtcctggctgttgaggaacccagtgtgagggtgctgagctatgccccaggtcccctggacaccaacatgcagcagttggcccgggaaacctccatggacccagagttgaggagcagactgcagaagttgaattctgagggggagctggtggactgtgggacttcagcccagaaactgctgagcttgctgcaaagggacaccttccaatctggagcccacgtggacttctatgacatttaataatgagtttgatccggctgctaacaaagcccgaaaggaagctgagttggctgctgccaccgctgagcaataactagcataaccccttggggcctctaaacgggtcttgaggggttttttgctgaaaggaggaactttcctggtttctggtcattgccaggcaggataaaacgtcgatcaacgctggcatgctctacttttttatcgcccacgccggatcggtgctgataatgatcgccttcttgctgatggggcgcgaaagcggcagcctcgattttgccagtttccgcacgctttcactttctccggggctggcgtcggcggtgttcctgctggatctcgatcccgcgaaattaatacgactcactataggggaattgtgagcggataacaattcccctctagaaataattttgtttaactttaagaaggagatatacatatggagagtgttccttggtttccaaagaagatttcagacctggaccattgtgctaaccgagttctgatgtatggatctgagctagatgcagaccaccctggcttcaaagacaatgtctaccgtaaaagacgaaagtactttgcagactcggctatgagctataaatatggagaccccattcctaaggttgaattcacggaagaggagattaagacctggggaaccgtattccgggagctcaacaaactctatccgacccatgcttgcagagagtatctcaaaaatttacctctgctttccaagtattgtggatatcaggaagacaatatcccacagctggaagatatttcaaactttttaaaagagcgcacaggtttttccattcgtcctgtggctggttacttatcaccaagagatttcttatcaggtttagcctttcgagtttttcactgcactcaatatgtgagacacagttcagaccccttctataccccagagccggatacctgccatgaactcttaggtcacgttccccttttggctgagccaagttttgctcagttctcccaagaaattggcctggcttcccttggagcttcagaggaggctgttcaaaaactggcaacgtgctactttttcactgtggagtttggtctatgtaaacaagacggacagttacgagtcttcggcgctggcttactttcttctatcagtgaactcaaacatgtgctttctggacatgccaaagtaaagccttttgatcccaagattacgtacaaacaagaatgcctcatcacaacttttcaggatgtctactttgtatctgaaagctttgaagatgcaaaggagaagatgagagaatttaccaaaacaattaagcgtccctttggagtgaaatataatccctacacacgaagcattcagatcctgaaagacgccaaaagctaataagccgcggaggattacactatggatatcatttctgtcgccttaaagcgtcattccactaaggcatttgatgccagcaaaaaacttaccccggaacaggccgagcagatcaaaacgctactgcaatacagcccatccagcaccaactcccagccgtggcattttattgttgccagcacggaagaaggtaaagcgcgtgttgccaaatccgctgccggtaattacgtgttcaacgagcgtaaaatgcttgatgcctcgcacgtcgtggtgttctgtgcaaaaaccgcgatggacgatgtctggctgaagctggttgttgaccaggaagatgccgatggccgctttgccacgccggaagcgaaagccgcgaacgataaaggtcgcaagttcttcgctgatatgcaccgtaaagatctgcatgatgatgcagagtggatggcaaaacaggtttatctcaacgtcggtaacttcctgctcggcgtggcggctctgggtctggacgcggtacccatcgaaggttttgacgccgccatcctcgatgcagaatttggtctgaaagagaaaggctacaccagtctggtggttgttccggtaggtcatcacagcgttgaagattttaacgctacgctgccgaaatctcgtctgccgcaaaacatcaccttaaccgaagtgtaataagccgcggaggattacactatgaaaacgacgcagtacgtggcccgccagcccgacgacaacggtttcatccactatccggaaaccgagcaccaggtctggaataccctgatcacccggcaactgaaggtgatcgaaggccgcgcctgtcaggaatacctcgacggcatcgaacagctcggcctgccccacgagcggatcccccagctcgacgagatcaacagggttctccaggccaccaccggctggcgcgtggcgcgggttccggcgctgattccgttccagaccttcttcgaactgctggccagccagcaattccccgtcgccacctttatccgcaccccggaagaactggactacctgcaggagccggacatcttccacgagatcttcggccactgcccactgctgaccaacccctggttcgccgagttcacccatacctacggcaagctcggcctcaaggcgagcaaggaggaacgcgtgttcctcgcccgcctgtactggatgaccatcgagttcggcctggtcgagaccgaccagggcaagcgcatctacggcggcggcatcctctcctcgccgaaggagaccgtctactgcctctccgacgagccgctgcaccaggccttcaatccgctggaggcgatgcgcacgccctaccgcatcgacatcctgcaaccgctctatttcgtcctgcccgacctcaagcgcctgttccaactggcccaggaagacatcatggcactggtccacgaggccatgcgcctgggcctgcacgcgccgctgttcccgcccaagcaggcggcctaataatgagtttgatccggctgctaacaaagcccgaaaggaagctgagttggctgctgccaccgctgagcaataactagcataaccccttggggcctctaaacgggtcttgaggggttttttgctgaaaggaggaactccatgcgctgttcaaagggctgctatttctcggcgcgggagcgattatttcgcgtttgcatacccacgacatggaaaaaatgggggcactagcgaaacggatgccgtggacagccgcagcatgcctgattggttgcctcgcgatatcagccattcctccgctgaatggttttatcagcgaatggtaccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatggcgcgccat

SEQ ID NO: 62 - DNA, 110 bp, Artificial
  Lac promoter
gagttagctcactcattaggcaccccaggctttacactttatgcttccggctcgtatgttgtgtggaattgtgagcggataacaatttcacacaggaaacagctatgacc

SEQ ID NO: 63 - DNA, 4920 bp, Artificial sequence
  Plasmid pBAD18kan
atcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgag

SEQ ID NO: 64 - DNA, 26 bp, Artificial sequence
  Primer Lin-pBAD-FWD
caactctctactgtttctccataccc

SEQ ID NO: 65 - DNA, 21 bp, Artificial sequence
  Primer Lin-pBAD-REV
gtttgcagaatccctgcttcg

SEQ ID NO: 66 - DNA, 41 bp, Artificial sequence
  Primer TPH-FWD
cgaagcagggattctgcaaaccaatacgcaaaccgcctctc

SEQ ID NO: 67 - DNA, 48 bp, Artificial sequence
  Primer TPH-REV
gggtatggagaaacagtagagagttgcaaatgccttagtggaatgacg

SEQ ID NO: 68 - DNA, 5271 bp, Artificial sequence
  Plasmid pTPH-H
atcgatgcataatgtgcctgtcaaatggacgaagcagggattctgcaaaccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatggatgacaaaggcaacaaaggcagcagcaaacgtgaagcggccaccgaaagcggcaaaaccgccgtggtttttagcctgaaaaacgaagtgggcggtctggtgaaagcgctgcgtctgtttcaggaaaaacgtgtgaacatggtgcatattgaaagccgtaaaagccgtcgccgtagcagcgaagtggaaatttttgtggattgcgaatgcggcaaaaccgaatttaacgaactgattcagctgctgaaatttcagaccaccattgtgaccctgaacccgccggaaaacatttggaccgaagaggaagagctggaagatgtgccgtggtttccgcgtaaaattagcgaactggataaatgcagccatcgtgtgctgatgtatggcagcgaactggatgcggatcatccgggctttaaagataacgtgtatcgtcagcgtcgcaaatattttgtggatgtggcgatgggctataaatatggccagccgattccgcgtgtggaatataccgaagaggaaaccaaaacctggggcgtggtttttcgtgaactgagcaaactgtatccgacccatgcgtgccgtgaatatctgaaaaactttccgctgctgaccaaatattgcggctatcgtgaagataacgtgccgcagctggaagatgtgagcatgtttctgaaagaacgtagcggctttaccgtgcgtccggtggcgggctatctgagcccgcgtgattttctggcgggcctggcgtatcgtgtgtttcattgcacccagtatattcgtcatggcagcgatccgctgtataccccggaaccggatacctgccatgaactgctgggccatgttccgctgctggccgatccgaaatttgcgcagtttagccaggaaattggcctggcgagcctgggcgcgagcgatgaagatgtgcagaaactggcgacctgctatttctttaccattgaatttggcctgtgcaaacaggaaggccagctgcgtgcctatggtgcgggcctgctgagcagcattggcgaactgaaacatgcgctgagcgataaagcgtgcgtgaaagcgtttgatccgaaaaccacctgcctgcaggaatgcctgattaccacctttcaggaagcgtattttgtgagcgaaagctttgaagaggcgaaagaaaaaatgcgaaaagcattacccgtccgtttagcgtgtattttaacccgtatacccagagcattgaaattctgaaagatacccgtagcattgaaaacgtggttcaggatctgcgtataataagccgcggaggattacactatggatatcatttctgtcgccttaaagcgtcattccactaaggcatttgcaactctctactgtttctccatacccgtttttttgggctagcgaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgag

SEQ ID NO: 69 - DNA, 5143 bp, Artificial sequence
  Plasmid pTPH-G
ctcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttcaagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcctgcaggtcgactctagaggatccccgggtaccgagctcgaattcgctagcccaaaaaaacgggtatggagaaacagtagagagttgcaaatgccttagtggaatgacgctttaaggcgacagaaatgatatccatagtgtaatcctccgcggcttattacatgacgtagctcattcaccacactggcaatgctcttggtgtctttcaggatctgcacactctgagtatacggattgtacttcacgccaaatggacgtttgatggtttttgcaaactctctcatcttttcctttgcttcttcaaaactttcagaaacaaagtaaacctcctggaaagttgtaatcaggcattcttgcttgcaggtgacctttggatcaaaaggcttgactttggcactgccagagagcgagtgcttgagctcactaatagaagagagcaggccagccccataaactctaagctgtccctcttgcttgcacaggccaaactctacagtgaaaaagtagcatgttgccagtttttggacagcctcgtctgatgccccaagtgatgcaagaccaatttcctgggagaactgagcaaaactgggttcagccaaaagagggacatggcctaggagctcatggcaggtatcaggctctggtgtgtagagagggtccgagctgtgtctaacatactgagtgcagtgaaaaactctgaatgctaatcctgccaagaagtctctgggtgacagatagccagcgactgggcgaatggtgaaacctgtgcgctctttcaggaagcgggacacgtcttccagctgggggatattgtcttccctgtacccacagtatttggtgagcaagggcaagtttttaaggtactctctgcaggcatgagttgggtaaagcttgttaagctctcggtatacagtcccccaagtcttgatctcctcctctgtgaattcaatctcgggaattgggtcaccatgcttgtagttcatagccaggtctgcaaaatactttcgcctcttacgatagacattgtctttgaaacctgggtggtcagcatccaaatcagacccgtacatcagcactcggtttgcacacttatccaaatctgagatcttctttggataccagggaatattctccatgtcaccatcctcctgcacattgaaatgctctgtcgggttcatagagacgatgctgacgtgggatttgaggagctggaagatctcattcagttgttccctattactgtcacagtcgacgaagatttcaaactccgagtttcgtctcttggatttccgtgactcgatgtgcacggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggtttgcagaatccctgcttcgtccatttgacaggcacattatgcatcgat

SEQ ID NO: 70 - DNA, 4941 bp, Artificial sequence
  Plasmid pTPH-OC
ctcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttcaagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcctgcaggtcgactctagaggatccccgggtaccgagctcgaattcgctagcccaaaaaaacgggtatggagaaacagtagagagttgcaaatgccttagtggaatgacgctttaaggcgacagaaatgatatccatagtgtaatcctccgcggcttattagcttttggcgtctttcaggatctgaatgcttcgtgtgtagggattatatttcactccaaagggacgcttaattgttttggtaaattctctcatcttctcctttgcatcttcaaagctttcagatacaaagtagacatcctgaaaagttgtgatgaggcattcttgtttgtacgtaatcttgggatcaaaaggctttactttggcatgtccagaaagcacatgtttgagttcactgatagaagaaagtaagccagcgccgaagactcgtaactgtccgtcttgtttacatagaccaaactccacagtgaaaaagtagcacgttgccagtttttgaacagcctcctctgaagctccaagggaagccaggccaatttcttgggagaactgagcaaaacttggctcagccaaaaggggaacgtgacctaagagttcatggcaggtatccggctctggggtatagaaggggtctgaactgtgtctcacatattgagtgcagtgaaaaactcgaaaggctaaacctgataagaaatctcttggtgataagtaaccagccacaggacgaatggaaaaacctgtgcgctcttttaaaaagtttgaaatatcttccagctgtgggatattgtcttcctgatatccacaatacttggaaagcagaggtaaatttttgagatactctctgcaagcatgggtcggatagagtttgttgagctcccggaatacggttccccaggtcttaatctcctcttccgtgaattcaaccttaggaatggggtctccatatttatagctcatagccgagtctgcaaagtactttcgtcttttacggtagacattgtctttgaagccagggtggtctgcatctagctcagatccatacatcagaactcggttagcacaatggtccaggtctgaaatcttctttggaaaccaaggaacactctccatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggtttgcagaatccctgcttcgtccatttgacaggcacattatgcatcgat

SEQ ID NO: 71 - DNA, 60 bp, Artificial sequence
  H1-P1-tnaA
atggaaaactttaaacatctccctgaaccgttccgcattcgtgtaggctggagctgcttc

SEQ ID NO: 72 - DNA, 59 bp, Artificial sequence
  H2-P2-tnaA
tcggttcgtacgtaaaggttaatcctttaatattcgccgcatatgaatatcctccttag

SEQ ID NO: 73 - DNA, 24 bp, Artificial sequence
  Primer tnaA-CFM-FWD
atctacaacagggcaaagcgcaac

SEQ ID NO: 74 - DNA, 25 bp, Artificial sequence
  Primer tnaA-CFM-REV
caccggcaagatcaacaggtaaagc

SEQ ID NO: 75 - DNA, 20 bp, Artificial sequence
  Primer K1
cagtcatagccgaatagcct

SEQ ID NO: 76 - DNA, 37 bp, Artificial sequence
  Primer THB-FWD
cacacaggaaaacatatgccatcactcagtaaagaag

SEQ ID NO: 77 - DNA, 35 bp, Artificial sequence
  Primer THB-REV
taaaaacggttagcgcagcaggaacaccgccgacg

SEQ ID NO: 78 - DNA, 3064 bp, Artificial sequence
  Plasmid pTH19Cr
atgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccataatgtgagttagctcactcattaggcaccccaggctttacactttatgcttccggctcgtatgttgtgtggaattgtgagcggataacaatttcacacaggaaaacat

SEQ ID NO: 79 - DNA, 26 bp, Artificial sequence
  Primer pTH19cr-Lin-FWD
cgctaaccgtttttatcaggctctgg

SEQ ID NO: 80 - DNA, 31 bp, Artificial sequence
  Primer pTH19cr-Lin-REV
atgttttcctgtgtgaaattgttatccgctc

SEQ ID NO: 81 - DNA, 43 bp, Artificial sequence
  Primer DP-FWD
cacacaggaaacagctatgaccatggatatcatttctgtcgcc

SEQ ID NO: 82 - DNA, 43 bp, Artificial sequence
  Primer DP-REV
gttgtaaaacgacggccagtgcggatcaaactcattattaggc

SEQ ID NO: 83 - DNA, 2686 bp, Artificial sequence
  Plasmid pUC18
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagcccattcgccattcaggctgcgcaactgttgggaagggcgatcggtgcgggcctcttcgctattacgccacgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 84 - DNA, 21 bp, Artificial sequence
  Primer linPUC18-FWD
cactggccgtcgttttacaac

SEQ ID NO: 85 - DNA, 22 bp, Artificial sequence
  Primer linPUC18-REV
ggtcatagctgtttcctgtgtg

SEQ ID NO: 86 - DNA, 43 bp, Artificial sequence
  Primer Lac-DP-FWD
ctttcctggtttctggtcattgcacgacaggtttcccgactgg

SEQ ID NO: 87 - DNA, 49 bp, Artificial sequence
  Primer Lac-DP-REV
gggtatggagaaacagtagagagttgaaactcattattaggccgcctgc

SEQ ID NO: 88 - DNA, 49 bp, Artificial sequence; identical to SEQ ID NO: 87
  Primer Lac-DP-REV

SEQ ID NO: 89 - DNA, 42 bp, Artificial sequence
  Primer Pa-THB-FWD
cgaagcagggattctgcaaactcttgaagacgaaagggcctc

SEQ ID NO: 90 - DNA, 43 bp, Artificial sequence
  Primer Pa-THB-REV
ccagtcgggaaacctgtcgtgcaatgaccagaaaccaggaaag

SEQ ID NO: 91 - DNA, 5352 bp, Artificial sequence
  Plasmid pBAD33
atcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttggggcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatc

SEQ ID NO: 92 - DNA, 8074 bp, Artificial sequence
  Plasmid pTHBDP
atcgatgcataatgtgcctgtcaaatggacgaagcagggattctgcaaactcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtgatcggtctgtcaaaaattaaccgcattgtgcagttctttgcccagcgtccgcaggtgcaggaacgtctgacgcagcaaattcttattgcgctacaaacgctgctgggcaccaataacgtggctgtctcgatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactaataagccgcggaggattacactatgaacgcggcggttggccttcggcgccgcgcgcgattgtcgcgcctcgtgtccttcagcgcgagccaccggctgcacagcccatctctgagtgctgaggagaacttgaaagtgtttgggaaatgcaacaatccgaatggccatgggcacaactataaagttgtggtgacaattcatggagagatcgatccggttacaggaatggttatgaatttgactgacctcaaagaatacatggaggaggccattatgaagccccttgatcacaagaacctggatctggatgtgccatactttgcagatgttgtaagcacgacagaaaatgtagctgtctatatctgggagaacctgcagagacttcttccagtgggagctctctataaagtaaaagtgtatgaaactgacaacaacattgtggtctacaaaggagaataataagccgcggaggattacactatggaaggaggcaggctaggttgcgctgtctgcgtgctgaccggggcttcccggggcttcggccgcgccctggccccgcagctggccgggttgctgtcgcccggttcggtgttgcttctaagcgcacgcagtgactcgatgctgcggcaactgaaggaggagctctgtacgcagcagccgggcctgcaagtggtgctggcagccgccgatttgggcaccgagtccggcgtgcaacagttgctgagcgcggtgcgcgagctccctaggcccgagaggctgcagcgcctcctgctcatcaacaatgcaggcactcttggggatgtttccaaaggcttcctgaacatcaatgacctagctgaggtgaacaactactgggccctgaacctaacctccatgctctgcttgaccaccggcaccttgaatgccttctccaatagccctggcctgagcaagactgtagttaacatctcatctctgtgtgccctgcagcccttcaagggctggggactctactgtgcagggaaggctgcccgagacatgttataccaggtcctggctgttgaggaacccagtgtgagggtgctgagctatgccccaggtcccctggacaccaacatgcagcagttggcccgggaaacctccatggacccagagttgaggagcagactgcagaagttgaattctgagggggagctggtggactgtgggacttcagcccagaaactgctgagcttgctgcaaagggacaccttccaatctggagcccacgtggacttctatgacatttaataatgagtttgatccggctgctaacaaagcccgaaaggaagctgagttggctgctgccaccgctgagcaataactagcataaccccttggggcctctaaacgggtcttgaggggttttttgctgaaaggaggaactttcctggtttctggtcattgcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatggatatcatttctgtcgccttaaagcgtcattccactaaggcatttgatgccagcaaaaaacttaccccggaacaggccgagcagatcaaaacgctactgcaatacagcccatccagcaccaactcccagccgtggcattttattgttgccagcacggaagaaggtaaagcgcgtgttgccaaatccgctgccggtaattacgtgttcaacgagcgtaaaatgcttgatgcctcgcacgtcgtggtgttctgtgcaaaaaccgcgatggacgatgtctggctgaagctggttgttgaccaggaagatgccgatggccgctttgccacgccggaagcgaaagccgcgaacgataaaggtcgcaagttcttcgctgatatgcaccgtaaagatctgcatgatgatgcagagtggatggcaaaacaggtttatctcaacgtcggtaacttcctgctcggcgtggcggctctgggtctggacgcggtacccatcgaaggttttgacgccgccatcctcgatgcagaatttggtctgaaagagaaaggctacaccagtctggtggttgttccggtaggtcatcacagcgttgaagattttaacgctacgctgccgaaatctcgtctgccgcaaaacatcaccttaaccgaagtgtaataagccgcggaggattacactatgaaaacgacgcagtacgtggcccgccagcccgacgacaacggtttcatccactatccggaaaccgagcaccaggtctggaataccctgatcacccggcaactgaaggtgatcgaaggccgcgcctgtcaggaatacctcgacggcatcgaacagctcggcctgccccacgagcggatcccccagctcgacgagatcaacagggttctccaggccaccaccggctggcgcgtggcgcgggttccggcgctgattccgttccagaccttcttcgaactgctggccagccagcaattccccgtcgccacctttatccgcaccccggaagaactggactacctgcaggagccggacatcttccacgagatcttcggccactgcccactgctgaccaacccctggttcgccgagttcacccatacctacggcaagctcggcctcaaggcgagcaaggaggaacgcgtgttcctcgcccgcctgtactggatgaccatcgagttcggcctggtcgagaccgaccagggcaagcgcatctacggcggcggcatcctctcctcgccgaaggagaccgtctactgcctctccgacgagccgctgcaccaggccttcaatccgctggaggcgatgcgcacgccctaccgcatcgacatcctgcaaccgctctatttcgtcctgcccgacctcaagcgcctgttccaactggcccaggaagacatcatggcactggtccacgaggccatgcgcctgggcctgcacgcgccgctgttcccgcccaagcaggcggcctaataatgagtttcaactctctactgtttctccatacccgtttttttgggctagcgaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttggggcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatc

SEQ ID NO: 93 - DNA, 5103 bp, Artificial sequence
  Plasmid pTHB
atgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtgatcggtctgtcaaaaattaaccgcattgtgcagttctttgcccagcgtccgcaggtgcaggaacgtctgacgcagcaaattcttattgcgctacaaacgctgctgggcaccaataacgtggctgtctcgatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactaataagccgcggaggattacactatgaacgcggcggttggccttcggcgccgcgcgcgattgtcgcgcctcgtgtccttcagcgcgagccaccggctgcacagcccatctctgagtgctgaggagaacttgaaagtgtttgggaaatgcaacaatccgaatggccatgggcacaactataaagttgtggtgacaattcatggagagatcgatccggttacaggaatggttatgaatttgactgacctcaaagaatacatggaggaggccattatgaagccccttgatcacaagaacctggatctggatgtgccatactttgcagatgttgtaagcacgacagaaaatgtagctgtctatatctgggagaacctgcagagacttcttccagtgggagctctctataaagtaaaagtgtatgaaactgacaacaacattgtggtctacaaaggagaataataagccgcggaggattacactatggaaggaggcaggctaggttgcgctgtctgcgtgctgaccggggcttcccggggcttcggccgcgccctggccccgcagctggccgggttgctgtcgcccggttcggtgttgcttctaagcgcacgcagtgactcgatgctgcggcaactgaaggaggagctctgtacgcagcagccgggcctgcaagtggtgctggcagccgccgatttgggcaccgagtccggcgtgcaacagttgctgagcgcggtgcgcgagctccctaggcccgagaggctgcagcgcctcctgctcatcaacaatgcaggcactcttggggatgtttccaaaggcttcctgaacatcaatgacctagctgaggtgaacaactactgggccctgaacctaacctccatgctctgcttgaccaccggcaccttgaatgccttctccaatagccctggcctgagcaagactgtagttaacatctcatctctgtgtgccctgcagcccttcaagggctggggactctactgtgcagggaaggctgcccgagacatgttataccaggtcctggctgttgaggaacccagtgtgagggtgctgagctatgccccaggtcccctggacaccaacatgcagcagttggcccgggaaacctccatggacccagagttgaggagcagactgcagaagttgaattctgagggggagctggtggactgtgggacttcagcccagaaactgctgagcttgctgcaaagggacaccttccaatctggagcccacgtggacttctatgacatttaataatgagtttgatccggctgctaacaaagcccgaaaggaagctgagttggctgctgccaccgctgagcaataactagcataaccccttggggcctctaaacgggtcttgaggggttttttgctgaaaggaggaactttcctggtttctggtcattgccaggcaggataaaacgtcgatcaacgctggcatgctctacttttttatcgcccacgccggatcggtgctgataatgatcgccttcttgctgatggggcgcgaaagcggcagcctcgattttgccagtttccgcacgctttcactttctccggggctggcgtcggcggtgttcctgctgcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccataatgtgagttagctcactcattaggcaccccaggctttacactttatgcttccggctcgtatgttgtgtggaattgtgagcggataacaatttcacacaggaaaacat

SEQ ID NO: 94 - DNA, 34 bp, Artificial sequence
  Primer GCH1-FWD
agtgcaggtaaaacaatgccatcactcagtaaag

SEQ ID NO: 95 - DNA, 26 bp, Artificial sequence
  Primer GCH1-REV
cgtgcgauttagttgtgatgacgcac

SEQ ID NO: 96 - DNA, 31 bp, Artificial sequence
  Primer PTPS-FWD
atctgtcataaaacaatgaacgcggcggttg

SEQ ID NO: 97 - DNA, 24 bp, Artificial sequence
  Primer PTPS-REV
cacgcgauttattctcctttgtag

SEQ ID NO: 98 - DNA, 31 bp, Artificial sequence
  Primer SPR-FWD
agtgcaggtaaaacaatggaaggaggcaggc

SEQ ID NO: 99 - DNA, 24 bp, Artificial sequence
  Primer SPR-REV
cgtgcgauttaaatgtcatagaag

SEQ ID NO: 100 - DNA, 33 bp, Artificial sequence
  Primer DHPR-FWD
agtgcaggtaaaacaatggatatcatttctgtc

SEQ ID NO: 101 - DNA, 25 bp, Artificial sequence
  Primer DHPR-REV
cgtgcgauttacacttcggttaagg

SEQ ID NO: 102 - DNA, 33 bp, Artificial sequence
  Primer PCBD1-FWD
atctgtcataaaacaatgaaaacgacgcagtac

SEQ ID NO: 103 - DNA, 24 bp, Artificial sequence
  Primer PCBD1-REV
cacgcgauttaggccgcctgcttg

SEQ ID NO: 104 - DNA, 33 bp, Artificial sequence
  Primer TPH-H-FWD
agtgcaggtaaaacaatggatgacaaaggcaac

SEQ ID NO: 105 - DNA, 27 bp, Artificial sequence
  Primer TPH-H-REV
cgtgcgauttatacgcagatcctgaac

SEQ ID NO: 106 - DNA, 32 bp, Artificial sequence
  Primer TPH-G-FWD
agtgcaggtaaaacagtgcacatcgagtcacg

SEQ ID NO: 107 - DNA, 24 bp, Artificial sequence
  Primer TPH-G-REV
cgtgcgauttacatgacgtagctc

SEQ ID NO: 108 - DNA, 33 bp, Artificial sequence
  Primer TPH-Oc-FWD
agtgcaggtaaaacaatggagagtgttccttgg

SEQ ID NO: 109 - DNA, 27 bp, Artificial sequence
  Primer TPH-OC-REV
cgtgcgauttagcttttggcgtctttc